(19) United States
(12) Patent Application Publication
(10) Pub. No.: US 2026/0191458 A1
(43) Pub. Date: Jul. 9, 2026

(54) SYSTEM AND METHODS FOR A LABOR COACH ALGORITHM

(71) Applicant: GE Precision Healthcare LLC, Waukesha, WI (US)

(72) Inventors: Kalaivani Manickam, Solihull (GB); Nagapriya Kavoori Sethumadhavan, Bengaluru (IN); Rajendra Naik, Bengaluru (IN)

(21) Appl. No.: 19/014,085

(22) Filed: Jan. 8, 2025

(51) Int. Cl. A61B 5/00; A61B 5/0205; A61B 8/02; A61B 8/08; G16H 50/70

(52) U.S. Cl. CPC A61B 5/4343; A61B 5/0205; A61B 5/486; A61B 5/7267; A61B 5/7278; A61B 5/7405; A61B 5/742; G16H 50/70; A61B 8/02; A61B 8/0866

(57) ABSTRACT

Methods and systems are provided for receiving labor-related data for one or more previous contractions of a patient, the labor-related data at least comprising data obtained with one of a tocodynamometer, an IUPC, and an EHG recording system, predicting labor-related data for a next contraction based on the labor-related data for one or more previous contractions, generating one or more visual indicators and one or more audio indicators based on output from a labor prediction model, the output from the labor prediction model being generated by entering the received labor-related data for the one or more previous contractions into the labor prediction model, the predicted labor-related data, and user input into the labor prediction model, and displaying the one or more visual indicators to a display device that is visible to the patient and outputting the one or more audio indicators to the patient.

### TECHNICAL FIELD

Embodiments of the subject matter disclosed herein relate to performing directed pushing by determining a desirable time duration for pushing based on output from a machine learning model or deep learning model that uses stored UA data and predicted UA data and displaying the desirable time duration via a user interface visible to the laboring mother.

### BACKGROUND

Uterine Activity (UA) corresponding to a mother's uterine contractions may be measured using a tocodynamometer, or toco, which measures a displacement of the mother's abdomen via a belt mounted mechanical pressure transducer. The UA may be represented as time-series data, which may be used to determine a frequency, an amplitude, a duration, and/or a period of the uterine contractions.

Uterine Activity (UA) acquired with a toco may be displayed to a clinician. In this way, the clinician may direct the laboring mother during each contraction based on the UA to ensure the mother pushes at a desirable time during the contraction. However, despite the directions from the clinician, the clinician may not provide instructions that lead to efficient pushing by a mother and the laboring mother may remain unclear with regards to the duration demanded for pushing for a particular contraction, which may result in perineal tearing due to delayed pushing or reduced pushing efficacy. The UA may also indicate that the contraction strength and frequency may adversely affect the mother and/or the fetal patient. As such, an alert may be provided to the clinician to mitigate adverse effects to the mother and/or fetal patient in response to contraction characteristics exceeding a pre-determined threshold, the pre-determined threshold being determined based on contractions, fetal and maternal parameters, and the maternal health history, including previous pregnancies, nature of previous pregnancies, maternal BP, and the like.

### SUMMARY

The current disclosure at least partially addresses one or more of the above identified issues via a method, comprising receiving labor-related data for one or more previous contractions of a patient, the labor-related data at least comprising data obtained with one of a tocodynamometer, an IUPC, and an EHG recording system, predicting labor-related data for a next contraction based on the labor-related data for one or more previous contractions, generating one or more visual indicators and one or more audio indicators based on the received labor-related data for the one or more previous contractions, the predicted labor-related data, and user input, and displaying the one or more visual indicators to a display device that is visible to the patient and outputting the one or more audio indicators to the patient.

The above advantages and other advantages, and features of the present description will be readily apparent from the Detailed Description below when taken alone or in connection with the accompanying drawings. It should be understood that the summary above is provided to introduce in simplified form a selection of concepts that are further described in the detailed description. It is not meant to identify key or essential features of the claimed subject matter, the scope of which is defined uniquely by the claims that follow the detailed description. Furthermore, the claimed subject matter is not limited to implementations that solve any disadvantages noted above or in any part of this disclosure.

### BRIEF DESCRIPTION OF THE DRAWINGS

Various aspects of this disclosure may be better understood upon reading the following detailed description and upon reference to the drawings in which:

FIG. 1 shows an exemplary tocodynamometer;

FIG. 2 is a schematic diagram of an exemplary UA detection system used to provide instructions to a patient during labor;

FIG. 3A is an example process for generating predicted labor-related data for a next contraction based on labor-related data for previous contractions;

FIG. 3B is an example process for generating visual indicators depicting instructions based on the labor-related data and predicted labor-related data;

FIG. 4 is an example process for training a labor prediction model based on labor-related data for one or more patients;

FIG. 5 is a flowchart illustrating a method for using a UA detection system to provide instructions to the patient during labor;

FIG. 6 is a flow chart illustrating a method for generating one or more visual indicators that provide instructions to a patient during labor;

FIG. 7 is a flow chart illustrating a method for displaying one or more visual indicators that provide instructions to a patient during labor;

FIG. 8 is a flow chart illustrating a method for updating the one or more visual indicators that provide instructions to the patient during labor;

FIG. 9 is a flow chart illustrating a method for training a labor prediction model based on labor-related data for one or more patients;

FIGS. 10-12 show example display outputs of a display device that is visible to the patient;

FIG. 13 shows a timing diagram wherein one or more visual indicators are updated during the progression of a contraction;

FIG. 14 shows an example process for training a contraction prediction model; and

FIG. 15 is a flow chart illustrating a method for training a contraction prediction model based on labor-related data for one or more patients; and

FIG. 16 is a flow chart illustrating a method for generating predicted labor-related data with the contraction prediction model.

### DETAILED DESCRIPTION

Ensuring a laboring mother is effectively pushing at appropriate times may lead to a healthy newborn and mother. Clinicians rely on uterine activity data acquired with and displayed with a monitor or printed on a strip chart, the monitor being coupled to one of a tocodynamometer (toco), an EHG recording system, and an IUPC respectively.

The toco sensor may be applied to the abdomen above the umbilicus where the fundal height of the uterus is palpated, and may be held to the upper part of the abdomen by an elastic belt. A heartbeat of the fetal patient may be monitored using a Doppler Ultrasound transducer, a Fetal Scalp Electrode, or an EMG patch during uterine contractions, while contraction detection allows monitoring of the progress of labor.

Uterine contractions can also be detected and/or measured using an electrohysterogram (EHG). Uterine contractions are the result of the coordinated actions of individual myometrial cells. At the cellular level, the contractions are triggered by a voltage signal called an action potential. During pregnancy, cellular electrical connectivity increases such that the action potential propagates to produce a coordinated contraction involving the entire uterus. The action potential during a uterine contraction can be measured with electrodes placed on the maternal abdomen resulting in a uterine EMG signal. The EHG signal can be processed to produce a signal that is similar to the standard uterine activity signal from the tocodynamometer or IUPC. In addition to contraction frequency and duration information, the EHG may provide an accurate indication of an intensity of a contraction.

Uterine contraction can also be detected and/or measured using an intrauterine pressure catheter (IUPC). The IUPC is positioned in an amniotic space (e.g., a uterus) during labor. A pressure transducer at an end of the catheter measures pressure changes in the uterus. The pressure changes are related to the strength, contraction, and duration of various contractions during labor.

Collectively, toco data acquired with the toco, the EHG data with an EHG recording system, or the IUPC data collected with the IUPC may be used to monitor a progression of labor, individual contractions, contraction duration, frequency, and strength, and the like. The clinician relies on the aforementioned information to direct a laboring mother. However, there may be discrepancies with regards to a timing of pushing by the laboring mother despite the clinician relaying instructions to the mother based on the information available to the clinician.

To address the problem of inappropriately timed pushing by a laboring mother, systems and methods are disclosed herein for generating one or more visual indicators and one or more audio indicators based on output from a labor prediction model that provides visual and auditory instruction to a patient during labor, the output from the labor prediction model being generated based on labor-related data for one or more previous contractions and predicted labor-related data for a next contraction, and displaying the one or more visual indicators to the patient, and outputting the one or more audio indicators to the patient.

The labor-related data may be classified as measured parameters or external (or associated) parameters. The measured parameters may include uterine activity (UA), fetal heart rate (FHR), maternal heart rate (MHR), trends of the measured parameters, and related parameters. The external parameters may include a history of previous C-sections, number of deliveries, blood pressure (BP), a number of accepted contractions, and the like. More specifically, the labor-related data may include UA data acquired from an IUPC, a tocodynamometer, or an EHG recording system. The labor-related data may include various labor parameters related to the current state of the patient, such as maternal HR, maternal BP, etc., medical history of the patient with regards to previous pregnancies, including whether the patient has had a C-section, whether the patient has had obstructed labor, tachysystole, PPH, preeclampsia, or other pregnancy or labor related complications, the current state of the fetal patient based on FHR and CTG, desired contraction characteristics, and the like.

The various labor parameters may define threshold values that are acceptable during a contraction. By including both measured parameters or external/associated parameters, the output from the labor prediction model and contraction prediction model described herein may be more accurate due to including medical parameters related to the unique medical history of a particular patient. In this way, the output data generated by the labor prediction model and the contraction prediction model is based on the unique medical history of the particular patient. In this way, more accurate instructions may be provided to the patient during labor that ensure that the health of the patient and the fetal patient are not adversely affected.

In this way, labor-related data regarding previous contractions may be used to predict labor-related data for a next contraction and any subsequent contractions. The labor-related data may be predicted with a contraction prediction algorithm or a contraction prediction model. The labor-related data and the predicted labor-related data may be used to predict a strength and duration of the next contraction and may be compared with the respective thresholds to evaluate potential consequences for the patient and the fetal patient during the next contraction.

One or more visual indicators may be generated based on output from a labor prediction model. The output from the labor prediction model may be generated by entering the labor-related data for the one or more previous contractions and the predicted labor-related data for the next contraction into the labor prediction model. The one or more visual indicators may be displayed to a display device visible to the patient. Further, one or more audio indicators may be generated based on the output from the labor prediction model as well. The one or more visual indicators may depict a progression of labor, a current extent of cervical dilation, directions prompting the patient, and a start time, a pushing time, and a resting time for the patient. By providing the patient with the visual indicators and the audio indicators, the labor of the patient may progress with reduced consequences for the health of the patient and the fetal patient since the visual indicators and the audio indicators may allow for more timely actions (e.g., pushing) by the patient. Further, prompting clinician intervention may alert the clinician to any potential complications during labor and may enable the clinician to intervene as demanded.

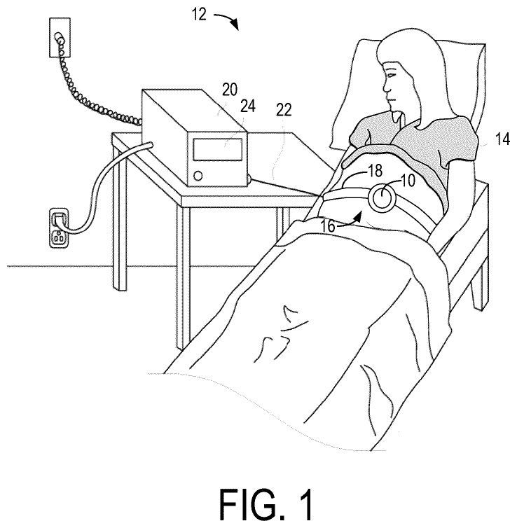

FIG. 1 is an environmental view of an exemplary embodiment of a maternal and fetal monitoring system 12 that includes use of a toco transducer 10, also referred to herein as toco 10, which may be used for simultaneously monitoring the health of a maternal patient 14 and fetal patient. Toco 10 is exemplarily secured to the abdomen 16 of the maternal patient 14, for example, by way of an elastomeric strap 18. However, it will be recognized that in other embodiments, a biocompatible adhesive may be used to secure the toco 10 to the patient's abdomen 16.

The toco 10 may be communicatively connected to a monitoring device 20 by a communicative connection 22, which may be a wired or a wireless communicative connection. Depending upon the configuration of the maternal and fetal monitoring system and the data transmitted between the toco 10 and the monitoring device 20, some or all of the data processing of the physiological information acquired the transducer may be performed locally at the toco 10. A controller located within the toco 10 may receive the acquired physiological data and process such physiological data in the manners as described herein and the UA may be communicated across the communicative connection 22 to the monitoring device 20 exemplarily for visual presentation on a graphical display 24 and/or electronic storage of this information on a data network of the hospital or medical facility and exemplarily in an electronic medical record (EMR) of the maternal patient.

In other embodiments, the toco 10 may perform more limited signal processing on the acquired physiological data and provide this physiological data across the communicative connection 22 to a monitoring device 20 which applies the signal processing actions and techniques as described herein to calculate UA.

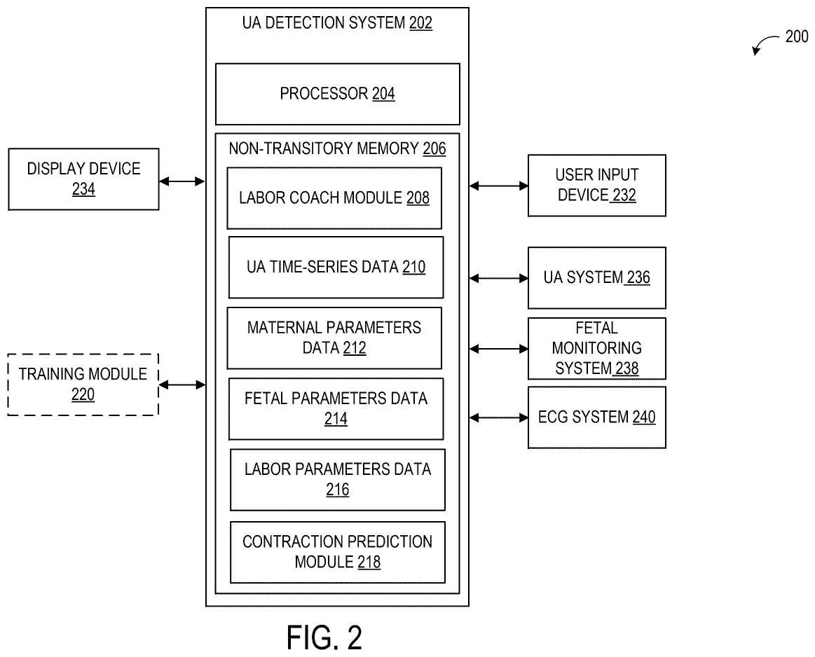

Referring now to FIG. 2, a block diagram 200 shows a UA detection system 202, in accordance with an embodiment. In some embodiments, UA detection system 202 may be incorporated into a medical system, such as a UA system. The UA system may be one of the toco of FIG. 1, an EHG recording system, or an IUPC. In some embodiments, at least a portion of UA detection system 202 is disposed at a device (e.g., edge device, server, etc.) communicably coupled to the UA system via wired and/or wireless connections. In some embodiments, UA detection system 202 may be operably/communicatively coupled to a user input device 232 and a display device 234.

UA detection system 202 may be operably/communicatively coupled to UA system 236 which may provide UA data, respectively. In this way, the UA data may be used for predicting a demanded pushing time for a subsequent contraction via executable instructions for a labor prediction model included in the labor coach module 208. UA detection system 202 may be operably/communicatively coupled to a fetal monitoring system 238 that monitors fetal heart rate (FHR). UA detection system 202 may be operably/communicatively coupled to an ECG system 240 to measure maternal heart rate (MHR). In embodiments wherein the UA system 236 is a tocodynamometer, the fetal monitoring system 238 may be a Doppler ultrasound system. In this way, the Doppler ultrasound system may be used to determine FHR. In embodiments wherein the UA system 236 is an IUPC, the fetal monitoring system 238 may be a FECG system. In this way, the FECG system may be used to determine FHR. In embodiments wherein the UA system 236 is an EHG recording system, there may be no additional fetal monitoring system 238 or ECG system 240 operably/communicatively coupled to the UA detection system 202. Instead, an EMG patch of the EHG recording system may perform FECG to determine FHR, MECG to determine MHR, and UA.

UA detection system 202 includes a processor 204 configured to execute machine readable instructions stored in non-transitory memory 206. Processor 204 may be single core or multi-core, and the programs executed thereon may be configured for parallel or distributed processing. In some embodiments, processor 204 may optionally include individual components that are distributed throughout two or more devices, which may be remotely located and/or configured for coordinated processing. In some embodiments, one or more aspects of processor 204 may be virtualized and executed by remotely-accessible networked computing devices configured in a cloud computing configuration.

Non-transitory memory 206 may store a labor coach module 208, UA time-series data 210, maternal parameters data 212, fetal parameters data 214, and labor parameters data 216, and a contraction prediction module 218. The labor coach module 208 stores executable instructions that causes the processor to receive UA time-series data from UA time-series data 210 for one or more previous contractions, receive maternal parameters data from maternal parameters data 212, receive fetal parameters data from fetal parameters data 214, and receive labor parameters data from labor parameters data 216. Each of the UA time-series data, the maternal parameters data, the fetal parameters data, and the labor parameters data may be considered labor-related data. The labor coach module 208 further stores instructions that utilizes the data received by the labor coach module to predict the characteristics (e.g., corresponding data) of a next contraction via the labor prediction model and executable instructions included in the contraction prediction module 218.

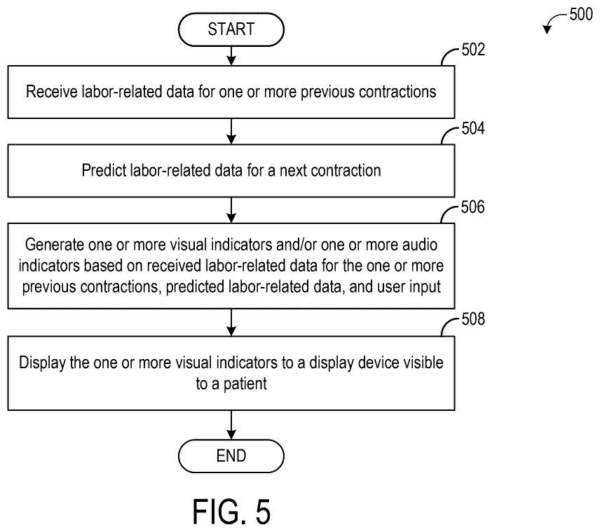

The labor coach module 208 may also store instructions that utilizes the predicted labor data for the next contraction and any subsequent contractions as well the labor data for the previous contraction to determine labor progression, a desired pushing time for the laboring mother, and instructions for directing the mother with regards to initiating pushing, terminating pushing, and prompting the patient to follow the instructions of a clinician based on output from the labor prediction model. The labor coach module 208 may also store instructions that generates one visual indicator that depicts the extent of cervical dilation based on user input received or data received from a digital cervimetry device. In particular, the labor coach module 208 may include instructions that, when executed by processor 204, cause UA detection system 202 to conduct one or more of the steps of method 500, method 600, method 700, and method 800 described in FIGS. 5-8.

Non-transitory memory 206 further stores UA time-series data 210. UA time-series data 210 may include physiological time-series data collected via various medical devices or systems, where the physiological time-series data includes indications of uterine contractions of a pregnant mother. For example, UA time-series data 210 may include UA data acquired via UA system 236. The UA system may be an IUPC, an EHG recording system, or tocodynamometer, as an example. UA time-series data 210 may include, for example, UA time-series data collected for the laboring mother in real-time. The UA time-series data 210 may include data collected for previous contractions during labor.

Each of the maternal parameters data 212, the fetal parameters data 214, and the labor parameters data 216 is considered labor-related data. As described herein, labor-related data may be classified as measured parameters or external (associated) parameters. Thus, a subset of each of the maternal parameters data 212, the fetal parameters data 214, and the labor parameters data 216 may be measured parameters and another subset of each of the maternal parameters data, the fetal parameters data, and the labor parameters data may be external (associated) parameters. Maternal parameters data 212 may include maternal heart rate (MHR), blood pressure (BP), a number of previous labors, previous C-sections, and the like. Fetal parameters data 214 may include fetal heart rate (FHR) during previous contractions, appropriate accelerations and decelerations of FHR, fetal movement, etc.

Labor parameters data 216 may include pre-determined conditions for contractions, such as an acceptable number of contractions per a pre-determined time duration, a number of contractions that were longer than expected and their respective time duration, a number of contractions that were stronger than expected, undesired rise times, and other contraction characteristics. Labor parameters data 216 may also include pre-determined conditions for predicted characteristics of the next contraction, such as anticipated duration of the next contraction, anticipated strength of the next contraction, anticipated issues during the next contraction (e.g. uterine rupture), etc.

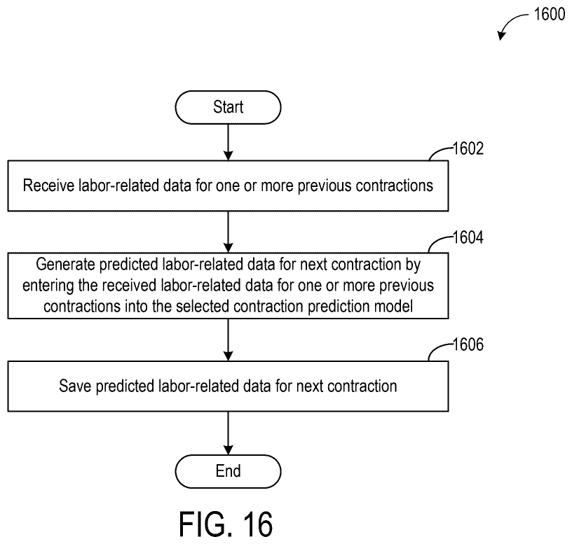

The labor coach module 208 and the contraction prediction module 218 may include one or more ML/DL models, and instructions for implementing the one or more ML/DL models to predict UA (e.g., uterine contractions) of a patient based on internal parameters including UA, FHR, MHR data, and the like and external parameters, such as maternal BP, cervical dilation, number of contractions, one set time of a first set of contractions, a number of previous C-section deliveries, etc. Contraction prediction module 218 may include models of various types, including trained and/or untrained neural networks such as Convolutional Neural Networks (CNNs), Recurrent Neural Networks (RNNs), statistical models, or other models, and may further include various data, or metadata pertaining to the one or more models stored therein. In particular, contraction prediction module 218 may include a contraction prediction model for predicting a UA of the patient based on UA data continuously collected during a preceding time-series window (e.g., during one or more previous contractions). In particular, the contraction prediction module 218 may include instructions that, when executed by processor 204, cause UA detection system 202 to conduct one or more of the steps of method 1600 in FIG. 16. In other embodiments, the contraction prediction module 218 may include conventional algorithms that do not include machine learning, such as averaging previous UA time-series data, extrapolation, etc. Alternatively, the contraction prediction module 218 may include instructions that predict an onset of a next contraction based on a current FHR trend, or rather, deceleration of the FHR.

UA detection system 202 may be operably/communicatively coupled to a training module 220, which may comprise instructions for training one or more of the models stored in the contraction prediction module 218 and the labor coach module 208. The training module 220 may include instructions that, when executed by a processor, cause the processor to build a model (e.g., a mathematical model) based on sample data to make predictions or decisions regarding UA time-series data without the explicit programming of a conventional algorithm that does not utilize machine learning. In one example, the training module 220 includes instructions for receiving training data sets from the UA time-series data 210.

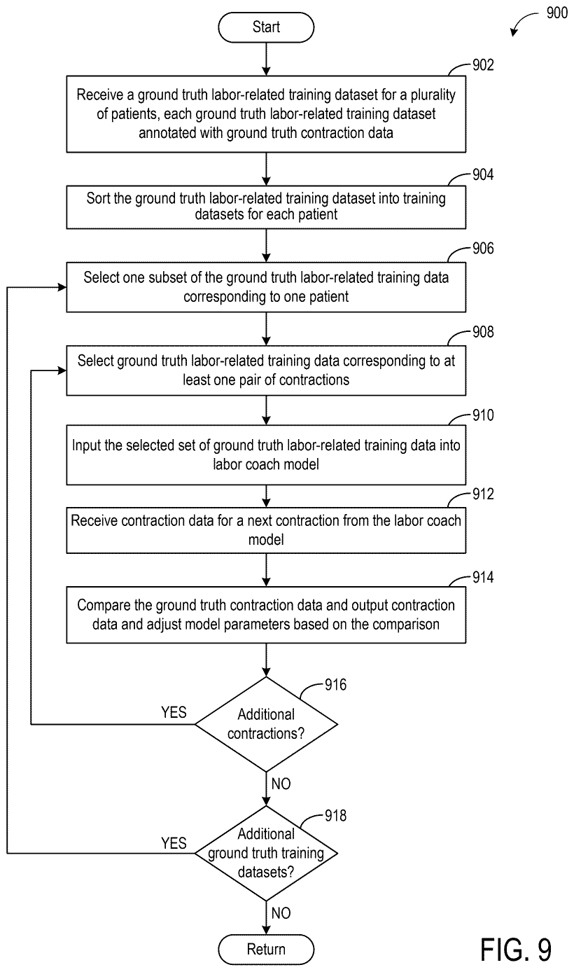

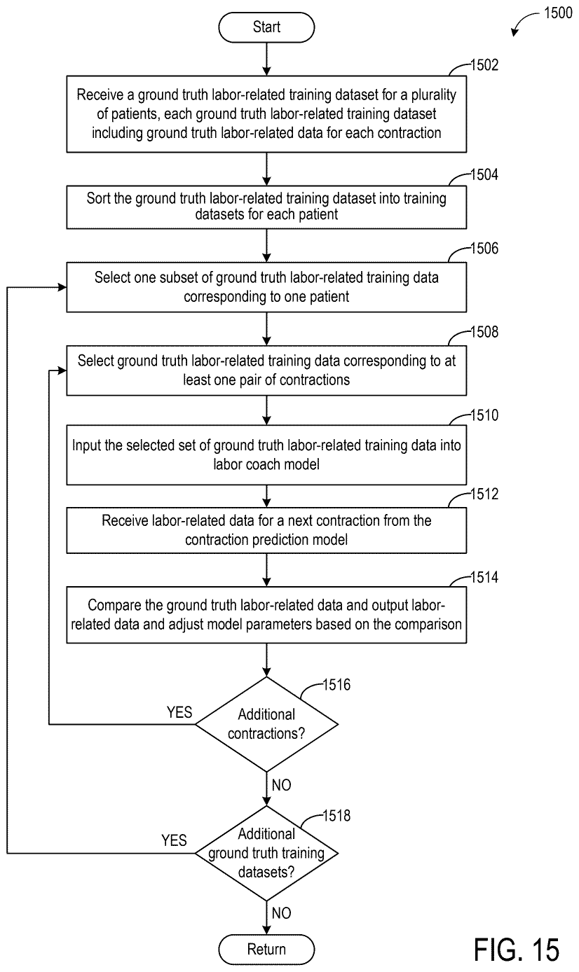

The training module 220 may include instructions, that when executed by a processor, cause the processor to build a model (e.g., a mathematical model) based on samples to make predictions or decisions regarding contraction data for a next contraction based on labor-related data without the explicit programming of a conventional algorithm that does not utilize machine learning. In particular, the training module 220 may include instructions that, when executed by processor 204, cause UA detection system 202 to conduct one or more of the steps of method 900 of FIG. 9 and method 1500 of FIG. 15. The training data sets comprise sets of datasets, associated ground truth labels/datasets, and associated model outputs for use in training one or more of the machine learning (ML) models or deep learning (DL) models stored in the contraction prediction module 218 and the labor coach module 208. For example, when the contraction frequency, duration of contraction, intensity mean area of the contractions, baseline values, etc. from the UA data and/or labor-related data are used for training, a ML model may be used. When the entire time series UA signal along with other maternal time series signals and fetal time series signals are used for training, a DL model (sequential network models) may be used.

In this way, the output from the contraction prediction model and the labor prediction model may account for the availability of different labor-related data or lack thereof. In this way, outputs from the contraction prediction model or labor prediction model generated based on more labor-related data may be more accurate than those generated based on less labor-related data. The training module 220 may receive datasets, associated ground truth labels/datasets, and associated model outputs for use in training the one or more machine learning models from sources other than the UA time-series data 210, such as the cloud, etc.

In some embodiments, one or more aspects of the training module 220 may include remotely-accessible networked storage devices configured in a cloud computing configuration. Further, in some embodiments, the training module 220 is included in the non-transitory memory 206. Additionally, or alternatively, in some embodiments, the training module 220 may be used to generate the contraction prediction module 218 and the labor coach module 208 offline and remote from the UA detection system 202. In such embodiments, the training module 220 may not be included in the UA detection system 202 but may generate data stored in the UA detection system 202. For example, the contraction prediction module 218 and the labor coach module 208 may be pre-trained with the training module 220 at a place of manufacture.

User input device 232 may comprise one or more of a touchscreen, a keyboard, a mouse, a trackpad, or other device configured to enable a user to interact with and manipulate data within UA detection system 202. The user input device 232 may enable a user to enter cervical dilation data and enter user input that overrides execution of instructions stored in the labor coach module and executes instructions stored in the labor coach module that instructs that patient to follow clinician directions.

Display device 234 may include one or more display devices utilizing virtually any type of technology. In some embodiments, display device 234 may comprise a computer monitor or a projector that projects the image on a wall or ceiling that is visible to the patient. Display device 234 may be combined with processor 204, non-transitory memory 206, and/or user input device 232 in a shared enclosure, or may be peripheral display devices and may comprise a monitor, touchscreen, projector, or other display device known in the art. The display device 234 may be included as part of a user interface (not shown) that displays a first visual indicator depicting a progression of labor, a second visual indicator depicting an extent of cervical dilation of the laboring mother, a third visual indicator depicting instructions to guide the laboring mother during a next contraction and subsequent contractions, and a fourth visual indicator depicting a remaining pushing time demanded for the next contraction during labor.

It should be understood that UA detection system 202 shown in FIG. 2 is for illustration, not for limitation. Another appropriate UA detection system may include more, fewer, or different components.

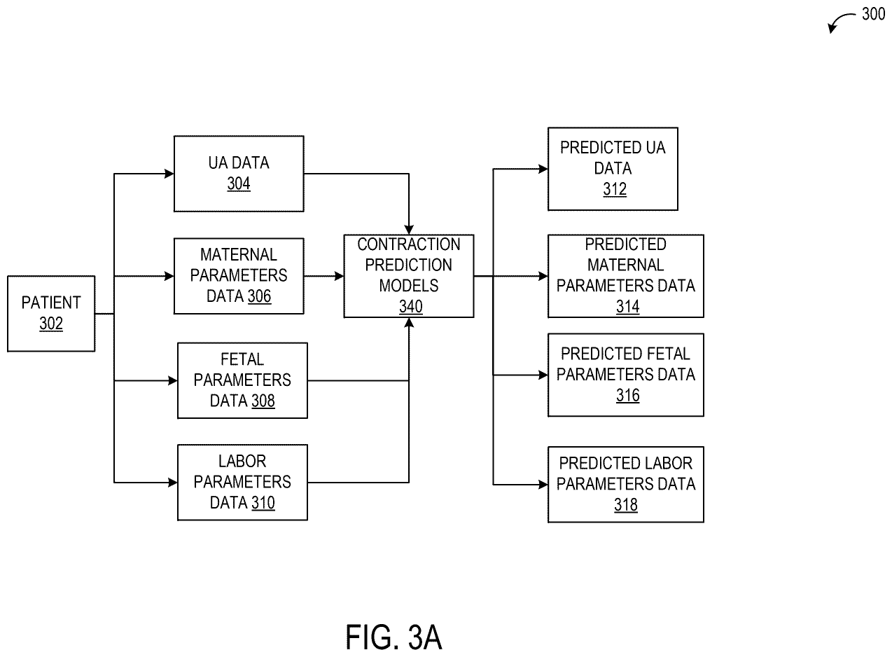

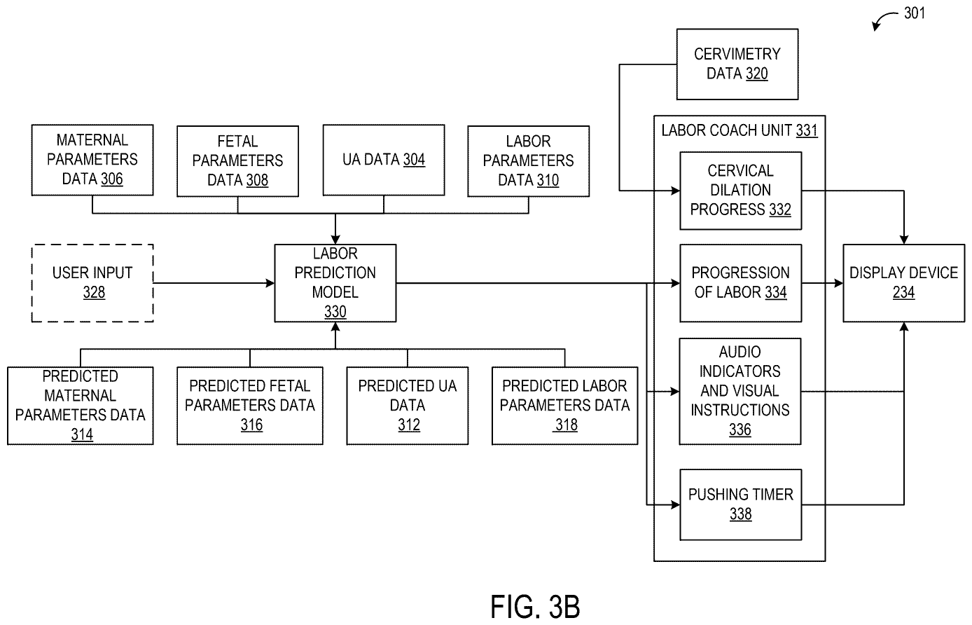

FIG. 3A illustrates a process 300 for predicting labor characteristics of a next contraction based on data pertaining to one or more previous contractions and FIG. 3B illustrates a process 301 for determining a current progression of labor, an extent of cervical dilation, and a demanded pushing time based on output from a labor prediction model, the output from the labor prediction model being generated by entering the data for the one or more previous contractions and the predicted data for the next contraction determined according to the process 300 detailed in FIG. 3A.

Turning to FIG. 3A, the process 300 may be performed by the UA detection system 202 of FIG. 2. The process 300 may include obtaining data from a patient 302 with various medical systems that monitor the patient during labor. For example, the various medical systems may include an IUPC, a tocodynamometer, or an EHG recording system communicatively coupled to the UA detection system 202 of FIG. 2. In this way, UA data for the one or more previous contractions may be obtained with one of the IUPC, the tocodynamometer, and the EHG recording system. In particular, IUPC data for the one or more previous contractions and a next contraction may be obtained with the IUPC. Similarly, toco data for the one or more previous contractions and the next contraction may be obtained with the tocodynamometer. Further, EHG data for the one or more previous contractions and the next contraction may be obtained with the EHG recording system.

The UA data 304 may be stored in the UA time-series data 210 of FIG. 2. While UA data for the patient 302 is described herein as being obtained with the IUPC, the tocodynamometer, and or EHG recording system, it is to be appreciated that data for the one or more previous contractions and the next contraction may be obtained with other types or additional medical systems.

The process 300 may include entering the UA data 304, maternal parameters data 306, fetal parameters data 308, and labor parameters data 310 for one or more previous contractions into one or more contraction prediction models 340. In some embodiments, the one or more contraction prediction models 340 may be machine learning (ML) or deep learning (DL) models. The one or more contraction prediction models 340 may be trained on UA data (e.g., IUPC data, toco data, and EHG data). In other embodiments, the one or more contraction prediction models 340 may be conventional algorithms that utilize data extrapolation, averaging, and the like to determine the predicted UA data 312 (e.g., predicted IUPC data, the predicted toco data, or the predicted EHG data), the predicted maternal parameters data 314, the predicted fetal parameters data 316, and the predicted labor parameters data 318 for the next contraction.

For example, IUPC data may be entered into the one or more contraction prediction models 340 along with other labor-related data for one or more previous contractions, toco data may be entered into the one or more contraction prediction models along with other labor-related data for one or more previous contractions, or the EHG data may be entered into the one or more contraction prediction models along with other labor-related for one or more previous contractions to generate predicted IUPC data, predicted toco data, and predicted EHG data, respectively, for the next contraction.

As described herein, the maternal parameters data 306, the fetal parameters data 308, and the labor parameters data 310 may be considered labor-related data. The labor-related data may be classified as measured parameters and external (associated) parameters. As such, each of the maternal parameters data 306, the fetal parameters data 308, and the labor parameters data 310 includes both measured parameters and external (associated) parameters. Accordingly, the measured parameters data of one or more previous contractions for each of the maternal parameters data 306, the fetal parameters data 308, and the labor parameters data 310 may be entered into the one or more contraction prediction models 340 along with the UA data 304 (e.g., one of IUPC data, toco data, and EHG data to generate predicted maternal parameters data 314 predicted fetal parameters data 316, and the predicted labor parameters data 318, respectively, for the next contraction. The one or more contraction prediction models 340 may be stored in the contraction prediction module 218 of FIG. 2. Thus, the process described above may be repeated for each contraction of the patient 302.

Turning to FIG. 3B, the process 301 may be performed by the UA detection system 202 of FIG. 2. The process 301 may include entering the acquired data for one or more previous contractions and the predicted data for the next contraction into a labor prediction model 330. The labor prediction model 330 may perform the method described herein with respect to FIGS. 5-8. The labor prediction model 330 may be trained on UA data, including IUPC data, toco data, and EHG data, and other labor-related data.

In particular, the process 301 may include entering the UA data 304, the maternal parameters data 306, the fetal parameters data 308, and the labor parameters data 310 into the labor prediction model 330. The process 301 may also include entering the predicted UA data 312, the predicted maternal parameters data 314, the predicted fetal parameters data 316, the predicted labor parameters data 318, and user input 328 into the labor prediction model 330. More specifically, the UA data 304, the predicted UA data 316, the maternal parameters data 306, the predicted maternal parameters data 314, the fetal parameters data 308, the predicted fetal parameters data 316, the labor parameters data 310, and the predicted labor parameters data 318 may be entered into the labor prediction model 330. Both measured parameters and external (associated parameters) included in the UA data 304, the predicted UA data 312, the maternal parameters data 306, the predicted maternal parameters data 314, the fetal parameters data 308, the predicted fetal parameters data 316, the labor parameters data 310, and the predicted labor parameters data 318 may be entered into the contraction prediction model 330.

In response to receiving data for one or more previous contractions and predicted data for the subsequent contraction, the labor prediction model 330, may output data regarding a progression of labor 334, and data for audio indicators and visual instructions 336, and data for a pushing timer 338. Cervimetry data 320 may be acquired using digital cervimetry and/or user input. The cervimetry data 320 used to determine cervical dilation progress 332, the data pertaining to the progression of labor 334, the audio indicators and visual instructions 336, and the pushing timer 338 may be processed by a labor coach unit 331. The labor coach unit 331 is communicatively coupled to a display device 234 and may be included in the labor coach module 208 of the UA detection system 202 of FIG. 2.

After being processed by the labor coach unit, the data pertaining to the cervical dilation progress 332, progression of labor 334, the audio indicators and visual instructions 336, and the pushing timer 338 may be output via speakers coupled to or integrated in the display device 234 or may be displayed as one or more visual indicators on a display device 234 to the patient 302. The pushing timer 338 includes data regarding a start time, a pushing duration, and an end time for pushing wherein the patient enters a resting time wherein the patient does not push.

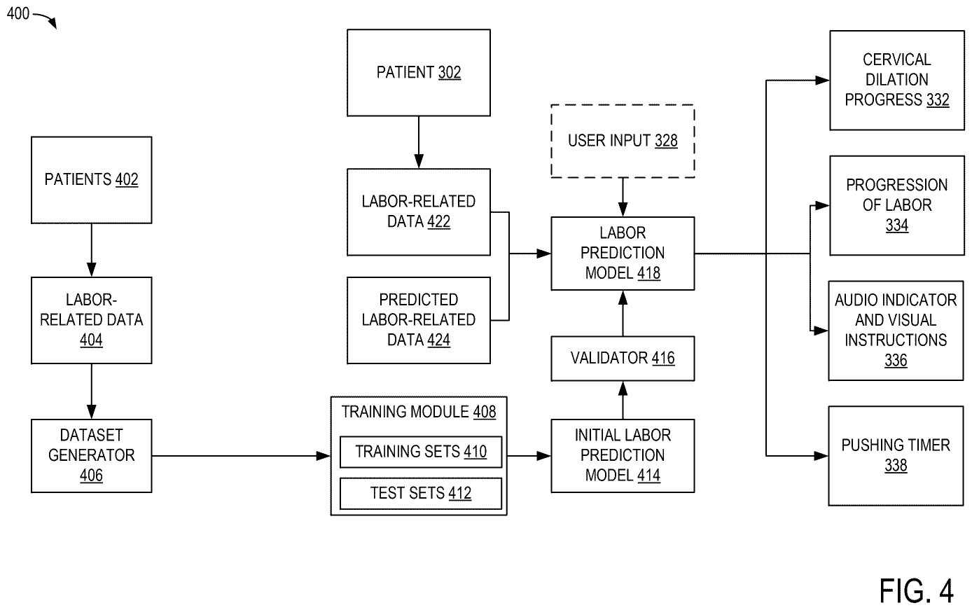

Turning to FIG. 4, a process 400 for training a labor prediction model 418 is illustrated. The labor prediction model 418 may be trained to identify contraction conditions conducive for pushing during labor and estimate various contraction parameters from labor-related data stored in an UA detection system, in accordance with one or more operations described in greater detail below in reference to FIG. 9. The labor prediction model 418 may be one or more ML/DL models or DL models. For example, the ML/DL models may include tree models and/or ensemble models and the DL models may include sequential RNN models and/or CNN models.

The labor prediction model 418 may be trained on UA data obtained with an IUPC (e.g., trained on IUPC data), UA data obtained with tocodynamometer (e.g., trained on toco data), UA data obtained with an EHG recording system (e.g., trained on EHG data), and other labor-related data. As described herein, contraction conditions may be conducive for pushing during labor when a likelihood of uterine rupture and adverse effects to the patient and fetal patient due to inappropriately timed pushing is reduced and the likelihood that labor progression is increased. Inappropriately timed pushing may include pushing when the vital signs of the patient and the fetal patient indicate physical distress.

The process 400 may be implemented by one or more computing systems, such as UA detection system 202 of FIG. 2, to train the labor prediction model 418 to identify contraction conditions conducive for pushing during labor and estimate various contraction parameters. Once trained, the labor prediction model 418 may be used to identify contraction conditions conducive for pushing during labor and estimating various contraction parameters for one or more patients during labor based on the labor-related data for a respective patient, in accordance with one or more operations described in greater detail below in reference to FIGS. 5-7.

The process 400 includes obtaining labor-related data 404 of a plurality of patients 402. For example, labor-related data may be obtained from a plurality of patients with different patient demographics, including different ages, different number of previous pregnancies, different number of previous C-sections, etc. The labor-related day may be classified as measured parameters and external (associated) parameters as described herein. The labor-related data 404 may include a plurality of subsets of the labor-related data 404. Each subset of labor-related data 404 may include labor-related data 404 for one patient and each subset of labor-related data may be ground truth labor-related data. The ground truth labor-related data of each subset of labor-related data 404 may indicate contraction conditions conducive for pushing, contraction conditions not conducive for pushing and conducive for clinician intervention, and estimated contraction parameters for each contraction conducive for pushing. As described herein with respect to FIGS. 2-3B, the labor-related data 404 may be UA data, maternal parameters data, fetal parameters data, and labor parameters data.

The process 400 includes generating a plurality of training sets of data using a dataset generator 406. The plurality of training sets of data may be stored in a training module 408. The training module 408 may be the same as or similar to the training module 220 of UA detection system 202 of FIG. 2. The plurality of training sets of data may be divided into training sets 410 and test sets 412. Each training set of training sets 410 and test sets 412 may include a subset of labor-related data 404 of one or more patients.

Once each set is generated, each set may be assigned to either the training sets 410 or the test sets 412. In an embodiment, the set may be assigned to either the training sets 410 or the test sets 412 randomly in a pre-established proportion (e.g., 90%/10% training/test, or 84%/14% training/test). It should be appreciated that the examples provided herein are for illustrative purposes, and sets may be assigned to the training sets 410 dataset or the test sets 412 dataset via a different procedure and/or in a different proportion without departing from the scope of this disclosure.

A number of training sets 410 and test sets 412 may be selected to ensure that sufficient training data is available to prevent overfitting, whereby an initial labor prediction model 414 learns to map features specific to samples of the training set that are not present in the test set. The process 400 includes training the initial labor prediction model 414 on the training sets 410. The process 400 may include a validator 416 that validates the performance of the initial labor prediction model 414 (as the initial model is trained) against the test sets 412. The validator 416 may take as input a trained or partially trained model (e.g., the initial labor prediction model 414, but after training and update of the model has occurred) and a dataset of test sets 412, and may output an assessment of the performance of the trained or partially trained labor prediction model on the dataset of test sets 412.

Thus, the initial labor prediction model 414 is trained on a training set wherein the training set includes a ground truth subset of labor-related data 404 for a particular patient. The initial labor prediction model 414 may be trained on UA data, including toco data, EHG data, and IUPC data, as well as other labor-related data. Additional training sets may be used to train the initial labor prediction model, each a subset of labor-related data 404, though it is to be appreciated that different subsets of labor-related data may be related to different patients.

The ground truth subset of labor-related data 404 may include ground truth contraction data for each contraction during labor. The ground truth contraction data may include a progression of labor, an extent of cervical dilation, and an estimated pushing time for each contraction, and the like. The ground truth contraction data may be compared with contraction data output from the initial labor prediction model 414, respectively, to calculate loss functions that are used to adjust model parameters of the initial labor prediction model.

In some examples, the labor prediction model 418 may be trained to identify contraction conditions conducive for pushing, identify contraction conditions conducive for clinician intervention, and to estimate contraction conditions. The contraction data output from the labor prediction model be used to generate visual indicators and audio indicators that may be output to a display device to instruct the patient during labor.

Once the validator 416 determines that the initial labor prediction model is sufficiently trained, the sufficiently trained initial labor prediction model (e.g., the labor prediction model 418) may be stored in the labor coach module 208 of FIG. 2. The labor prediction model 418, when deployed, may identify contraction data conducive for pushing, identify contraction data conducive for clinician intervention (e.g., not conducive for pushing based on output from the labor prediction model), and estimate contraction data (e.g., pushing time, etc.). In this way, visual indicators and audio indicators may be generated based on the estimated contraction data as described in FIGS. 5-9.

Newly-acquired labor-related data 422 obtained from a patient and predicted labor-related data 424 may be entered as input to the labor prediction model 418 to output contraction data that may be used to generate visual indicators and audio indicators for a progression of labor 334, a cervical dilation progress 332, audio indicator and visual instructions 336, and a pushing timer 338. Collectively, the visual indicators and audio indicators direct the patient during labor. In some examples, the visual indicators and audio indictors direct the patient to follow instructions of a clinician, provide instructions for pushing during labor, and provide instructions to cease pushing and entering a resting time.

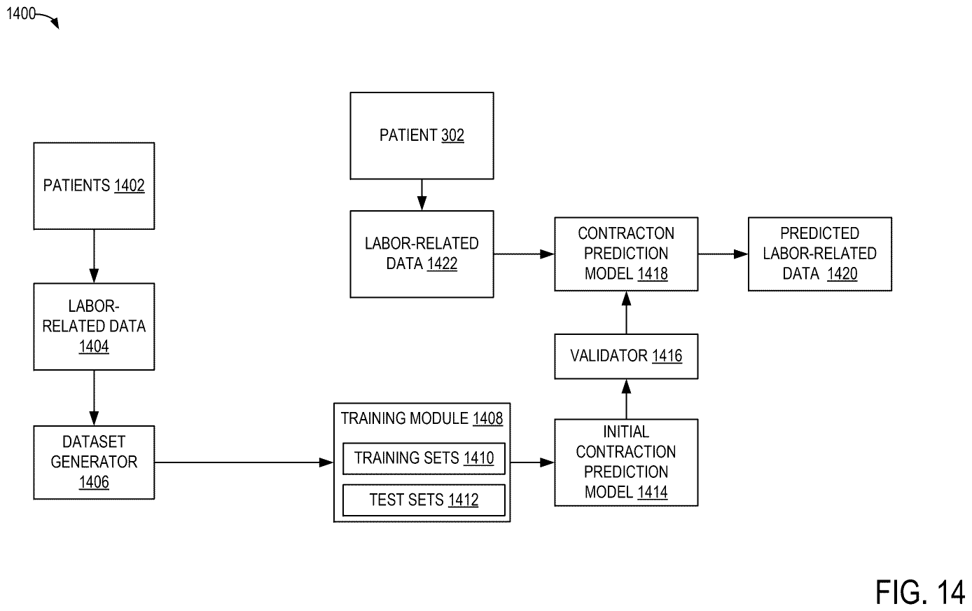

FIG. 14 illustrates a process 1400 for training a contraction prediction model 1418. The contraction prediction model 1418 may be trained to predict labor-related data (e.g., UA data and other labor-related data) for a next contraction based on labor-related data for one or more previous contractions, in accordance with one or more operations described in greater detail below in reference to FIG. 15. The contraction prediction model 1418 may be one or more ML/DL models. The contraction prediction model 1418 may be trained on UA data obtained with an IUPC (e.g., trained on IUPC data), UA data obtained with a toco (e.g., trained on toco data), UA data obtained with an EHG recording system (e.g., trained on EHG data), and other labor-related data.

The process 1400 may be implemented by one or more computing systems, such as UA detection system 202 of FIG. 2, to train the contraction prediction model 1418 to predict labor-related parameters for a next contraction. Once trained, the contraction prediction model 1418 may be used to predict labor-related data based on the labor-related data for a respective patient during one or more previous contractions, in accordance with one or more operations described in greater detail below in reference to FIG. 16.

The process 1400 includes obtaining labor-related data 1404 of a plurality of patients 1402. For example, labor-related data may be obtained from a plurality of patients with different patient demographics, including different ages, different number of previous pregnancies, different number of previous C-sections, etc. The labor-related data 1404 may include a plurality of subsets of the labor-related data 1404. Each subset of labor-related data 1404 may include labor-related data 1404 for one patient and each subset of labor-related data may be ground truth labor-related data. The ground truth subset of labor-related data 1404 may indicate contraction parameters of the next contraction, such as contraction strength, contraction duration, and contraction frequency. As described herein with respect to FIGS. 2-3B, the labor-related data 1404 may be UA data, maternal parameters data, fetal parameters data, and labor parameters data.

The process 1400 includes generating a plurality of training sets of data using a dataset generator 1406. The plurality of training sets of data may be stored in a training module 1408. The training module 1408 may be the same as or similar to the training module 220 of UA detection system 202 of FIG. 2. The plurality of training sets of data may be divided into training sets 1410 and test sets 1412. Each training set of training sets 1410 and test sets 1412 may include a subset of labor-related data 1404 of one or more patients.

Once each set is generated, each set may be assigned to either the training sets 1410 or the test sets 1412. In an embodiment, the set may be assigned to either the training sets 1410 or the test sets 1412 randomly in a pre-established proportion (e.g., 90%/10% training/test, or 84%/14% training/test). It should be appreciated that the examples provided herein are for illustrative purposes, and sets may be assigned to the training sets 1410 dataset or the test sets 1412 dataset via a different procedure and/or in a different proportion without departing from the scope of this disclosure.

A number of training sets 1410 and test sets 1412 may be selected to ensure that sufficient training data is available to prevent overfitting, whereby an initial contraction prediction model 1414 learns to map features specific to samples of the training set that are not present in the test set. The process 1400 includes training the initial contraction prediction model 1414 on the training sets 1410. The process 1400 may include a validator 1416 that validates the performance of the initial contraction prediction model 1414 (as the initial model is trained) against the test sets 1412. The validator 1416 may take as input a trained or partially trained model (e.g., the initial contraction prediction model 1414, but after training and update of the model has occurred) and a dataset of test sets 1412, and may output an assessment of the performance of the trained or partially trained contraction prediction model on the dataset of test sets 1412.

Thus, the initial contraction prediction model 1414 is trained on a training set wherein the training set includes a ground truth subset of labor-related data 1404 for a particular patient. The initial contraction prediction model 1414 may be trained on UA data, including toco data, EHG data, and IUPC data, as well as other labor-related data. Additional training sets may be used to train the initial contraction prediction model, each a subset of labor-related data 1404, though it is to be appreciated that different subsets of labor-related data may be related to different patients.

The ground truth subset of labor-related data may include the ground truth labor-related data for each contraction during labor. The ground truth labor-related data may include a strength for a particular contraction, a duration of the particular contraction, and a frequency of the particular contraction, etc. The ground truth labor-related data may be compared with labor-related data output from the initial contraction prediction model 1414, respectively, to calculate loss functions that are used to adjust model parameters of the initial contraction prediction model.

In some examples, the contraction prediction model 1418 may be trained to predict labor-related data for a next contraction. The labor-related data output from the contraction prediction model 1418 be entered as input to the labor prediction model as described herein with respect to FIG. 3B, and 5-8.

Once the validator 1416 determines that the initial contraction prediction model 1414 is sufficiently trained, the sufficiently trained initial contraction model (e.g., the contraction prediction model 1418) may be stored in the labor coach module 208 of FIG. 2. The contraction prediction model 1418, when deployed, may output predicted labor-related data 1420 for a next contraction based on UA data and labor-related data from one or more previous contractions.

Newly-acquired labor-related data 1422 obtained from the patient 302 may be entered as input to the contraction prediction model 1418 to output predicted labor-related data 1420, which includes at least one of predicted toco data, predicted IUPC data, and predicted EHG data, predicted maternal data, predicated maternal parameters data, predicted fetal parameters data, and predicted labor parameters data.

Referring now to FIG. 15, a flowchart is shown of a method 1500 for training a contraction prediction model. The contraction prediction model may be a non-limiting example of the contraction prediction model 1418 of the process 1400 of FIG. 14, according to an embodiment. In some embodiments, the contraction prediction model may be one or more machine learning (ML) models and/or deep learning (DL) models. Method 1500 may be executed by a processor of a UA detection system, such as the UA detection system 202 of FIG. 2. Method 1500 may be carried out according to instructions stored in non-transitory memory of the UA detection system (e.g., in a training module such as the training module 220 of FIG. 2) and executed by a processor of the UA detection system (e.g., the processor 204 of UA detection system 202 of FIG. 2).

The contraction prediction ML/DL model may be trained on training data comprising one or more sets of labor-related data for different patients. Each set of the one or more sets of labor-related data may include labor-related data for a plurality of contractions during labor, as described below. In some embodiments, the one or more sets of labor-related data may be stored in a labor-related database of an UA detection system, such as the UA time-series data 210, the maternal parameters data 212, the fetal parameters data 214, and the labor parameters data of UA detection system 202 of FIG. 2, which may include the UA data quantity parameter(s).

At 1502, the method 1500 includes receiving ground truth labor-related training dataset, the ground truth labor-related dataset being including ground truth labor-related data for each next contraction. The ground truth labor-related training dataset may include UA training data (e.g., toco training data, EHG training data, IUPC training data, etc.)) for a plurality of patients and other labor-related training data for the plurality of patients. The other labor-related training data may include number of previous pregnancies for each patient, number of C-sections for each patient, MHR and BP during each contraction for each patient, FHR during each contraction for each patient, rise times for each contraction for each patient, and the like. The ground truth labor-related training dataset may include labels indicating whether the UA data is intermittent and unusable, whether the UA data is intermittent and usable, whether the labor-related data is conducive for pushing, whether the labor-related data is conducive for clinician intervention, and estimated contraction data including progression of labor, extent of cervical dilation, and a pushing time for the next contraction. The ground truth labor-related training dataset may be stored the UA detection system (e.g., FIG. 2).

At 1504, the method 1500 includes sorting the ground truth labor-related dataset into datasets for each patient. As described above, the ground truth labor-related training dataset comprises data collected for a plurality of patients during labor. Each subset of ground truth labor-related training data includes training data pertaining to one patient during labor. In one embodiment, each subset of ground truth labor-related training data may correspond to a different patient.

At 1506, the method 1500 includes selecting one subset of ground truth labor-related training data corresponding to one patient. Each ground truth labor-related training dataset for a patient includes ground truth labor-related training data for each contraction during labor. For example, for each contraction, the ground truth labor-related training dataset may include ground truth labor-related training data for the next contraction. For example, the ground truth labor-related training data for a contraction (e.g., the most recent or current contraction) may include UA training data, maternal parameters training data, fetal parameters training data, and labor parameters training data for the next contraction.

At 1508, the method 1500 includes selecting ground truth labor-related training data corresponding to at least one pair of contractions. In one example, the at least one pair contractions may be one pair of contractions wherein one contraction is considered the previous contraction and the other contraction is considered the next contraction. In other examples, the at least one pair of contractions may be one or more contractions wherein at least one contraction is considered the next contraction and two or more contractions are considered the previous contractions. Instructions configured, stored, and executed in memory by a processor may cause the processor to randomly select the contractions. Alternatively, instructions configured, stored, and executed in memory by a processor may cause process to select the contractions based on a time stamp.

At 1510, the method 1500 includes inputting the selected set of ground truth labor-related training data into the contraction prediction ML/DL model. The contraction prediction ML/DL model may include one or more ML/DL models. The contraction prediction ML/DL is trained on UA data (e.g., toco data, EHG data, and IUPC data). In this way, depending on the type of UA data available, contraction data may be estimated using different types of UA data with the contraction prediction ML/DL model.

At 1512, the method 1500 includes receiving labor-related data from the contraction prediction ML/DL model. The contraction prediction ML/DL model may output a set of contraction data for a next contraction included in the ground truth labor-related training dataset.

At 1514, the method 1500 includes comparing the ground truth labor-related data and output labor-related data and adjusting model parameters of the contraction prediction ML/DL model. More specifically, the ground truth labor-related data of the ground truth labor-related training dataset may be compared with the set of labor-related data (e.g., for the next contraction) output from the contraction prediction ML/DL model. In an example, a loss function of the contraction prediction ML/DL model may be determined based on the ground truth labor-related data of the ground truth labor-related training dataset and the first set of output labor-related data for the next contraction. As such, the loss function of the contraction prediction ML/DL model may be used to update the parameters of the contraction prediction ML/DL model.

At 1516, the method 1500 includes determining whether additional contractions remain in the ground truth labor-related training data. The plurality of contractions included in the ground truth labor-related training data may include a pre-determined number of next contractions. If less than all of the pre-determined number of next contractions have been selected and used to train the contraction prediction ML/DL model (e.g., at least some contractions and corresponding contraction data remain), or if the contraction prediction ML/DL model is otherwise determined to not be fully trained, method 1500 returns to 1508 to select ground truth labor-related training data corresponding to a next at least one pair of contractions and use the next contraction and corresponding labor-related training data to train the contraction prediction ML/DL model. However, if at 1516 it is determined that each contraction and corresponding labor-related training data has been selected and used to train the contraction prediction ML/DL model (and no more contractions and corresponding labor-related training data remain), or if the contraction prediction ML/DL model is otherwise determined to be fully trained, method 1500 proceeds to 1518, which includes determining whether additional subsets of ground truth labor-related training data remain.

The plurality of subsets of labor-related data may include a pre-determined number of subsets of labor-related data for a plurality of patients. If less than all of the pre-determined number of subsets have been selected and used to train the contraction prediction ML/DL model (e.g., at least some subsets remain), or if the contraction prediction ML/DL model is otherwise determined to not be fully trained, method 1500 returns to 1506 to select a next subset and use the next subset to train the contraction prediction ML/DL model. However, if at 1518 it is determined that each subset has been selected and used to train the contraction prediction ML/DL model (and no more subsets remain), or if the contraction prediction ML/DL model is otherwise determined to be fully trained, the method 1500 then returns.

FIG. 16 illustrates a method 1600 for generating predicted labor-related data with one or more contraction prediction models. The method 1600 described herein may be executed by a processor of a UA detection system, such as UA detection system 202 of FIG. 2. Operations of the method 1600 may be stored in non-transitory memory of the UA detection system (e.g., in a contraction prediction module of the UA detection system 202 of FIG. 2) and executed by a processor of the UA detection system (e.g., processor 204).

At 1602, the method 1600 includes receiving labor-related data for one or more previous contractions. The labor-related data for one or more previous contractions includes UA data obtained with one of an IUPC, a tocodynamometer, and an EHG recording device. The labor-related data for the one or more previous contractions further comprises maternal parameters data, fetal parameters data, and labor parameters data. The maternal parameters data may include a number of previous pregnancies, a number of previous C-sections, maternal heart rate (MHR), blood pressure, and the like. The fetal parameters data includes fetal heart rate (FHR), fetal movement and the like.

At 1604, the method 1600 includes generating predicted labor-related data for a next contraction by entering the received labor-related data for one or more previous contractions into the contraction prediction model. As such, in response to receiving UA data and other labor-related data for one or more previous contractions, the contraction prediction ML/DL model generates predicted UA data and predicted labor-related data for the next contraction.

At 1606, the method 1600 includes saving the predicted labor-related data. The predicted labor-related data may be saved in UA times-series data 210, maternal parameters data 212, fetal parameters data 214, and labor parameters data 216 of the UA detection system 202 described in FIG. 2. In this way, the predicted labor-related data may be accessed by the labor coach module 208 of the UA detection system as described herein with regards to FIGS. 5-7. The method 1600 then ends.

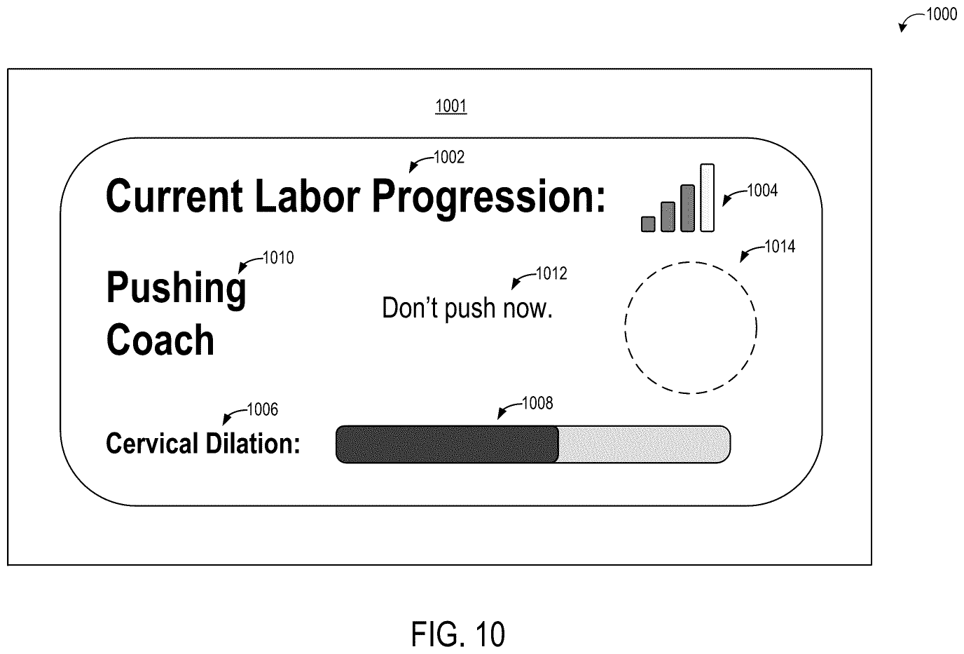

FIGS. 10-12 show example display outputs of a user interface 1001. For example, each display output may be output on a display device, such as the display device 234 of FIG. 1. The user interface 1001 includes a first label 1002, a second label 1006, and a third label 1010. The user interface 1001 also includes at least a first visual indicator 1004, a second visual indicator 1008, and a third visual indicator 1012. The user interface 1001 may further include a fourth visual indicator 1014. The first label 1002 is positioned next to the first visual indicator 1004 and the first label textually describes the information depicted in the first visual indicator.

For example, the first visual indicator 1004 may depict a current progression of labor relative to a total progression of labor. The second label 1006 is positioned next to the second visual indicator 1008 and the second label textually describes the information depicted in the second visual indicator. The second visual indicator 1008 may depict a current extent of cervical dilation relative to the desired extent of cervical dilation for giving birth. The third label 1010 is positioned next to the third visual indicator 1012 such that when the fourth visual indicator 1014 is displayed, the third visual indicator 1012 is positioned between the third label 1010 and the fourth visual indicator 1014.

The third label 1010 may textually describe a function of the third visual indicator 1012 and the fourth visual indicator 1014. The third visual indicator 1012 may be written text that provides instructions to the patient whereas the fourth visual indicator 1014 may be a pushing timer that visually depicts and textually describes a starting time for pushing, a remaining time left for pushing, and an end to the remaining pushing time and a start to a pushing break for the patient. Alternatively, the fourth visual indicator may be an image that does not visually depict or textually describe instructions for a patient.

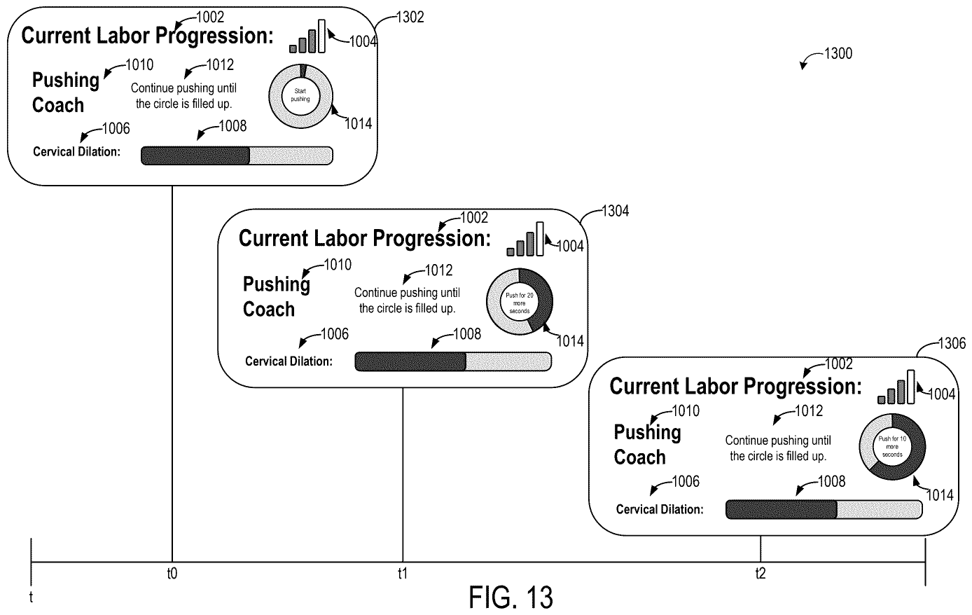

It may be understood that the arrangement of the user interface 1001, and more specifically, the arrangement first label 1002, the second label 1006, the third label 1010, the first visual indicator 1004, the second visual indicator 1008, the third visual indicator 1012, and the fourth visual indicator 1014 may differ from the examples provided in FIGS. 10-12 without departing from the scope of the present disclosure. For example, the arrangement of the user interface 1001 may include more or fewer labels and/or visual indicators in some embodiments so long as the arrangement displays instructions to the patient that lead to the patient pushing at appropriate times. In one example, the display may include a pushing timer that counts down as a contraction progresses. An example of the pushing timer counting down as the contraction progresses is illustrated in FIG. 13. In another example, the display may include a countdown timer that depicts an amount of time remaining before pushing is demanded by the patient, such as during a resting time. Further, the shapes and images of the first visual indicator 1004, the second visual indicator 1008, and the fourth visual indicator 1014 may differ from the examples provided without departing from the scope of the present disclosure.

FIG. 10 shows a first example of a display output 1000 of the user interface 1001. As shown in the display output 1000, the first visual indicator 1004 shows three filled bars out of four total bars. As such, the first visual indicator 1004 indicates that the progression of labor is at around 75% completion based on various parameters. The second visual indicator 1008 is a horizontal bar that is over half-way filled, indicating that the extent of cervical dilation is less than halfway from the desired extent of cervical dilation to give birth. Further, the third visual indicator 1012 shows written text prompting the patient to not push and the fourth visual indicator 1014 shows a circle formed by a dashed line without written text. The display output 1000 may be output in response to the end of the previous contraction. In other embodiments, the display 1000 may include a countdown timer that depicts a remaining time until pushing is demanded by the patient.

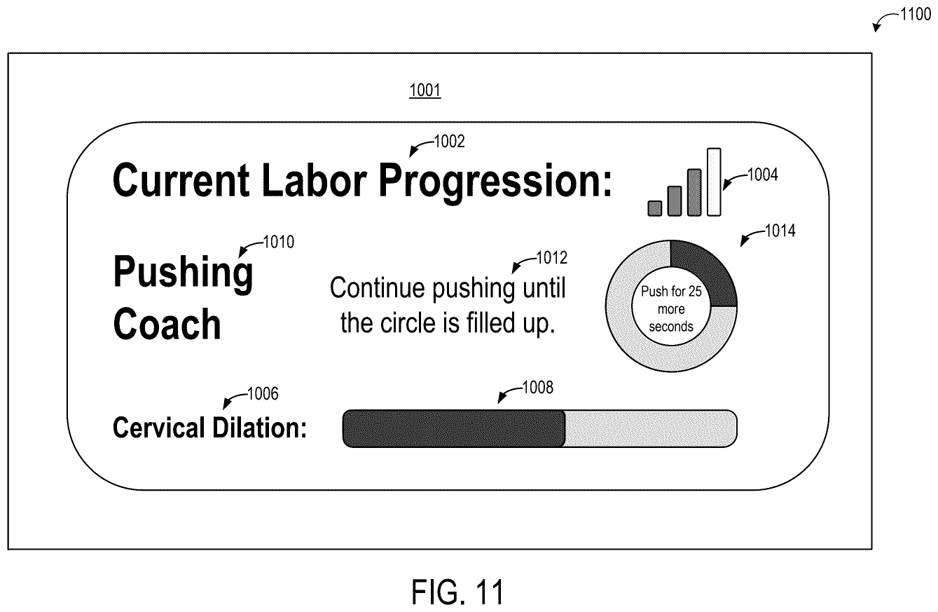

FIG. 11 shows a second example of a display output 1100 of the user interface 1001. As shown in the display output 1100, the first visual indicator 1004 shows three filled bars out of four total bars. As such, the first visual indicator 1004 indicates that the progression of labor is at around 75% completion based on various parameters. The second visual indicator 1008 is a horizontal bar that is over half-way filled, indicating that the extent of cervical dilation is less than halfway from the desired extent of cervical dilation to give birth. Further, the third visual indicator 1012 shows written text prompting the patient to push until a certain point (e.g., until the circle is filled up) and the fourth visual indicator 1014 shows a pushing timer denoted by a circle/ring-shape and written text that provides instruction to the patient. In particular, the instructions prompt the patient to push for a remaining amount of time. The display output 1100 may be output to the display device during the progression of the contraction.

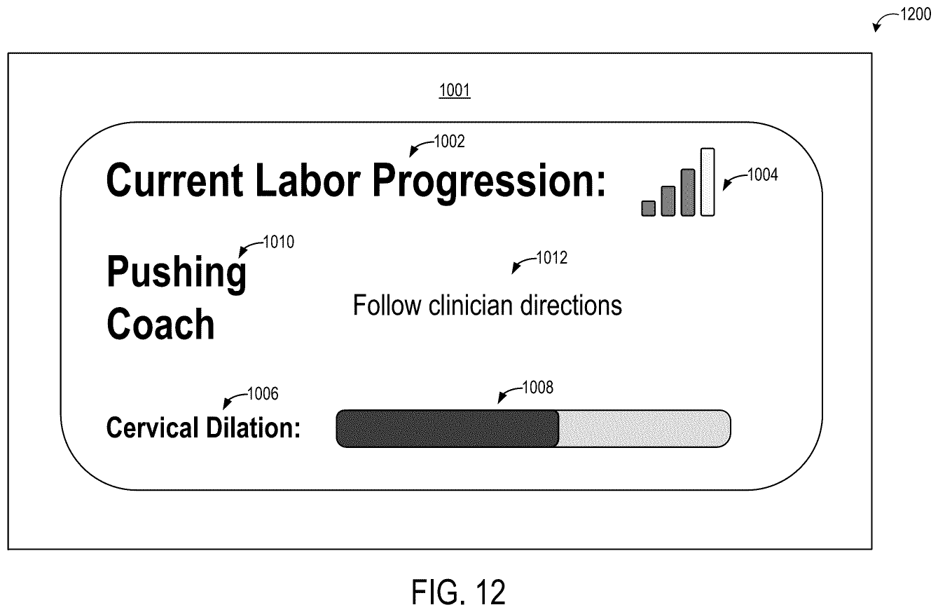

FIG. 12 shows a third example of a display output 1200 of the user interface 1001. As shown in the display output 1200, the first visual indicator 1004 shows three filled bars out of four total bars. As such, the first visual indicator 1004 indicates that the progression of labor is at around 75% completion based on various parameters. The second visual indicator 1008 is a horizontal bar that is over half-way filled, indicating that the extent of cervical dilation is less than halfway from the desired extent of cervical dilation to give birth. Further, the third visual indicator 1012 shows written text prompting the patient to follow the instructions of the clinician and the fourth visual indicator 1014 is no longer displayed on the display output 1200. The display output 1200 may be output to the display device before a contraction and during a contraction.

FIG. 5 illustrates a method 500 for directing a patient during labor with visual indicators displayed on a display device. The method 500 described herein may be executed by a processor of a UA detection system, such as UA detection system 202 of FIG. 2. Operations of the method 500 may be stored in non-transitory memory of the UA detection system (e.g., in a labor coach module of the UA detection system 202 of FIG. 2) and executed by a processor of the UA detection system (e.g., processor 204).

At 502, the method 500 includes receiving labor-related data for one or more previous contractions. The labor-related data for one or more previous contractions includes UA data as described herein. The labor-related data for the one or more previous contractions further comprises maternal parameters data, fetal parameters data, and labor parameters data. The maternal parameters data may include a number of previous pregnancies, a number of previous C-sections, maternal heart rate (MHR), blood pressure, and the like. The fetal parameters data includes fetal heart rate (FHR). The labor parameters data may include an acceptable number of contractions per pre-determined time duration, an acceptable contraction duration, an acceptable contraction strength, unacceptable rise times for a contraction, and indication that labor is progressing.

At 504, the method 500 includes predicting labor-related data for a next contraction. The labor-related data may be predicted based on the labor-related data for one or more previous contractions. In particular, the predicted labor-related data may be generated by entering the labor-related data into a contraction prediction model as depicted in FIG. 3A or contraction prediction algorithm. The contraction prediction algorithm may use techniques, such as averaging, or extrapolation based on the labor-related data of the one or more previous contractions. The contraction prediction model may be one or more ML/DL models trained on UA time-series data obtained with one of the IUPC, the tocodynamometer, or the EHG recording system, and other labor-related data according to embodiments described herein.

At 506, the method 500 includes generating one or more visual indicators and one or more audio indicators based on the received labor-related data for the one or more previous contractions, the predicted labor-related data, and user input. The one or more visual indicators may be generated by determining contraction data, including a progression of labor, an extent of cervical dilation, and determining an anticipated pushing time, an anticipated start time for pushing, and an anticipated end time for pushing based on the received labor-related data for the one or more previous contractions, the predicted labor-related data, and user input. In particular, the contraction data may be generated by entering the received labor-related data for the one or more previous contractions, the predicted labor-related data, and user input into labor prediction model. Upon receiving output (e.g., contraction data) from the labor prediction model, a first visual indicator is generated that depicts the progression of labor, a second visual indicator is generated that depicts the extent of cervical dilation, and a third visual indicator is generated that updates in real-time and displays instructions for the patient during labor.

Depending on the labor-related data for the one or more previous contractions and the predicted-labor data for the next contraction, a fourth visual indicator may be generated based on output from the labor prediction model as well. The fourth visual indicator may update in real-time and depict the start time for pushing, a remaining time for pushing, and a resting time for the patient. The first visual indicator, the second visual indicator, the third visual indicator, and the fourth visual indicator may be generated with a labor coach unit and/or instructions stored in labor coach module 208 of the UA detection system 202. Examples of the first visual indicator, the second visual indicator, and the third visual indicator are described herein with respect to FIGS. 10-12.

At 508, the method 500 includes displaying the one or more visual indicators to a display device that is visible to the patient and outputting the one or more audio indicators to the patient. By displaying the one or more visual indicators to the display device and outputting the one or more audio indicators to the patient, the patient may witness progression of labor and may push in a more appropriate manner based on the visual cues provided by the first visual indicator, the second visual indicator, the third visual indicator, and the fourth visual indicator when displayed. The method 500 then ends. It may be understood that the method described in FIG. 5 may be performed for subsequent contractions.

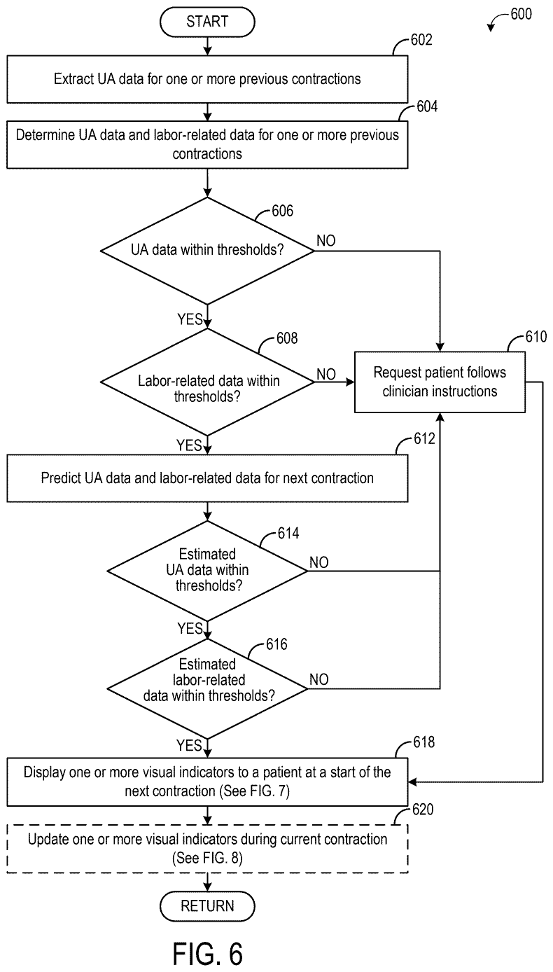

FIG. 6 illustrates a method 600 for generating one or more visual indicators based on UA data and labor-related data for one or more previous contractions and predicted UA data and labor-related data. The method 600 described herein may be executed by a processor of a UA detection system, such as UA detection system 202 of FIG. 2. Operations of the method 600 may be stored in non-transitory memory of the UA detection system (e.g., in a labor coach module of the UA detection system 202 of FIG. 2) and executed by a processor of the UA detection system (e.g., processor 204).

At 602, the method 600 includes extracting UA data for one or more previous contractions. The UA data may be extracted from UA time-series data collected during labor. In particular, the UA data may be extracted for the most recent contraction(s) from among all the UA time-series data collected.

At 604, the method 600 includes determining UA data and other labor-related data for one or more previous contractions. The UA data is considered labor-related parameters as described herein. More specifically, the UA data includes one or more of IUPC data, toco data, and EHG data. The UA data may be processed to determine a strength and a duration of each contraction and the frequency of the contractions. Further, the other labor-related data comprises the maternal parameters data, the fetal parameters data, and the labor-related data described herein.

At 606, the method 600 includes determining whether the UA data is within UA data thresholds. The UA data may be compared to UA data thresholds. The UA data thresholds may include information regarding acceptable values for the UA data. For example, the UA data thresholds may include acceptable contraction strength, acceptable contraction duration, acceptable frequency of multiple contractions, an acceptable number of contractions per pre-determined time duration, indicators that labor is progressing, acceptable rise times for the UA data, and the like for the one or more previous contractions. By including UA data thresholds, proper instructions may be provided to the patient during both normal labor progression and abnormal labor progression that may affect the health of the patient and the fetal patient.

In response to the extracted UA data not being within UA data thresholds, the method 600 includes requesting the patient to follow clinician instructions at 610. The extracted UA data not being within the UA data thresholds may indicate that the progression of labor is abnormal and predictions for a next contraction may be unreliable. Further, it may indicate that the health patient or fetal patient is experiencing adverse effects. As such, the clinician assesses the state of the patient based on the UA data for the next contraction and provides instructions to the patient during the next contraction.

At 618, the method 600 includes displaying one or more visual indicators at the start of the next contraction. At least a first visual indicator for a progression of labor, a second visual indicator for an extent of cervical dilation, and a third visual indicator that updates in real-time and relays a message to the patient may be generated based on the received UA data. The third visual indicator, in particular, may prompt the patient to follow the instructions of the clinician during the next contraction. An example of the third visual indicator prompting the patient to follows instructions of the clinician is depicted in FIG. 12. The third visual indicator, in particular, may prompt the patient to follow the instructions of the clinician during the next contraction. The at least first visual indicator, the second visual indicator, and the third visual indicator may be displayed on a display device visible to the patient. The method 600 then returns.

In response to the extracted UA data being within UA data thresholds, the method 600 includes determining whether the labor-related data is within labor-related data thresholds at 608. The other labor-related data may be compared to labor-related data thresholds. The labor-related data thresholds may include information regarding acceptable values for the other labor-related data. For example, the labor-related data thresholds may include acceptable maternal heart rate (MHR), maternal blood pressure (BP) acceptable fetal heart rate (FHR), acceptable accelerations of FHR, acceptable decelerations of FHR, and labor-related data threshold values indicating potential for uterine obstruction. By including labor-related data thresholds, proper instructions may be provided to the patient during both normal labor progression and abnormal labor progression that may affect the health of the patient and the fetal patient.

In response to the other labor-related data not being within labor-related data thresholds, the method 600 includes requesting the patient to follow clinician instructions at 610. The other labor-related data not being within the labor-related data thresholds may indicate that the progression of labor is abnormal and predictions for a next contraction may be unreliable. Further, it may indicate that the health patient or fetal patient is experiencing adverse effects. As such, the clinician assesses the state of the patient based on the UA data for the next contraction and provides instructions to the patient during the next contraction.

At 618, the method 600 includes displaying one or more visual indicators at a start of the next contraction. At least the first visual indicator, the second visual indicator, and the third visual indicator may be generated based on the extracted UA data. The third visual indicator, in particular, may prompt the patient to follow the instructions of the clinician during the next contraction. An example of the third visual indicator prompting the patient to follows instructions of the clinician is depicted in FIG. 12. The third visual indicator, in particular, may prompt the patient to follow the instructions of the clinician during the next contraction. The at least first visual indicator, the second visual indicator, and the third visual indicator may be displayed on a display device visible to the patient. The method 600 then returns.

In response to the labor-related data being within labor-related data thresholds, the method 600 includes predicting UA data and labor-related data for the next contraction at 612. As described herein with respect to FIG. 3A, predicted UA data and predicted labor-related data for the next contraction may be generated by a contraction prediction algorithm that leverages extrapolation and averaging of one or more previous contractions or a contraction prediction model trained on UA data and/or labor-related data.

At 614, the method 600 includes determining whether the predicted UA data is within UA data thresholds. The predicted UA data for the next contraction may be compared to UA data thresholds. As described herein, the UA data thresholds may include information regarding acceptable values for the UA data.

In response to the predicted UA data not being within UA data thresholds, the method 600 includes requesting the patient to follow clinician instructions at 610. For example, the UA data thresholds for the next contraction may be based on an acceptable contraction strength for the next contraction, an acceptable contraction duration for the next contraction, indication of labor progression, acceptable patient vitals (e.g., BP and MHR) for a next contraction with the predicted strength and predicted duration, potential (or probability) for uterine rupture based on factors including primigravida, multigravida, previous C-sections, etc. Similar to the extracted UA data not being within the UA data thresholds, the predicted UA data not being within the UA data thresholds may also indicate risk to maternal and/or fetal patient, or that the progression of labor is abnormal and/or that the predictions for the next contraction may be unreliable. In this way, the patient may not receive proper instructions for labor events wherein the contraction strength and duration is unmanageable for the physical state of the patient and the fetal patient. As such, the clinician assesses the state of the patient based on the UA data for the next contraction and provides instructions to the patient during the next contraction.

At 618, the method 600 includes displaying one or more visual indicators at the start of the next contraction. At least the first visual indicator, the second visual indicator, and the third visual indicator may be generated based on the extracted UA data, the other labor-related data, and the predicted UA data. The third visual indicator, in particular, may prompt the patient to follow the instructions of the clinician during the next contraction. The at least first visual indicator, the second visual indicator, and the third visual indicator may be displayed on a display device visible to the patient. The method 600 then returns.

In response to the predicted UA data being within the UA data thresholds, the method 600 includes determining whether the predicted labor-related data is within labor-related data thresholds at 616. The predicted labor-related data may be compared to labor-related data thresholds. The labor-related data thresholds may include information regarding acceptable values for the labor-related data. For example, the labor-related data thresholds may include an acceptable FHR based on FHR patterns during previous contractions and the predicted contraction strength and duration of the next contraction. By including labor-related data thresholds, proper instructions may be provided to the patient during both normal labor progression and abnormal labor progression that may affect the health of the patient and the fetal patient.

In response to the predicted labor-related data not being within labor-related data thresholds, the method 600 includes requesting the patient to follow clinician instructions at 610. The predicted labor-related data not being within the labor-related data thresholds may indicate risk to the maternal and/or fetal patient, or that the progression of labor is abnormal and/or that the predictions for a next contraction may be unreliable. As such, the clinician assesses the state of the patient based on the UA data for the next contraction and provides instructions to the patient during the next contraction.

At 618, the method 600 includes displaying one or more visual indicators at the start of the next contraction. At least the first visual indicator, the second visual indicator, and the third visual indicator may be generated based on the extracted UA data, the other labor-related data, the predicted UA data or onset of current UA data combined with predicted UA data, and the predicted labor-related data. The third visual indicator, in particular, may prompt the patient to follow the instructions of the clinician during the next contraction. The at least first visual indicator, the second visual indicator, and the third visual indicator may be displayed on a display device visible to the patient. The method 600 then returns.

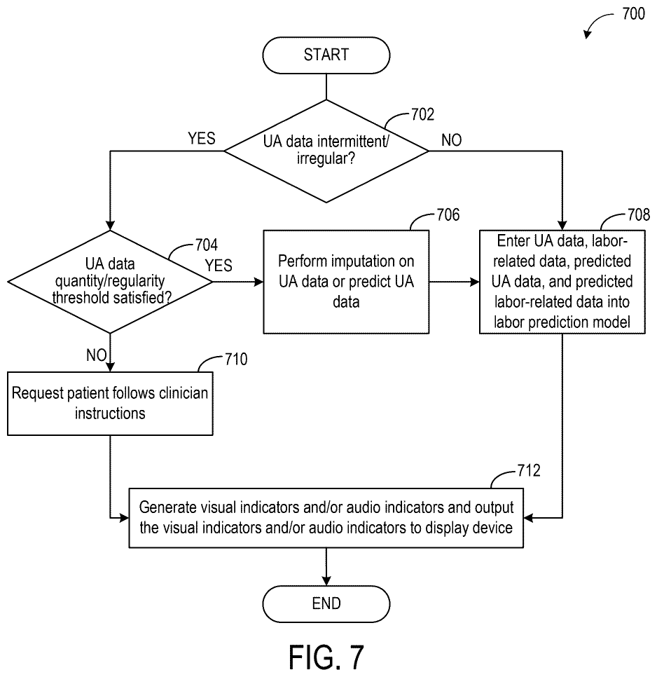

In response to the predicted labor-related data being within the labor-related data thresholds, the method 600 includes displaying one or more visual indicators at the start of the next contraction at 618. The first visual indicator, the second visual indicator, the third visual indicator, and a fourth visual indicator depicting instructions for the patient may be generated based on the UA data, the labor-related data, the predicted UA data, and the predicted labor-related data. More specifically, the first visual indicator, the second visual indicator, and the third visual indicator may be generated based on output from the labor prediction model according to FIG. 7, the output being generated in response to entering the UA data, the labor-related data, the predicted UA data, and the predicted labor-related data into the labor prediction model. The fourth visual indicator may update in real-time and may be displayed to the patient to depict a start time for pushing during labor. Further, a first audio indicator may be generated based on output from the labor prediction model and output to the patient to alert the patient to begin pushing in response to the UA data thresholds and the labor-related thresholds being satisfied.

Accordingly, the third visual indicator may update based on the fourth visual indicator. For example, prior to the beginning of the next contraction, the third visual indicator may prompt the patient to either follow instructions of the clinician as shown in FIG. 12 or instruct the patient to not push to allow the patient to have a resting time as shown in FIG. 10. In response to the fourth visual indicator being generated (or updated), the message depicted by the third visual indicator updates to prompt the patient to push as shown in FIG. 11. An example of the third visual indicator updating based on the fourth visual indicator is illustrated in FIG. 13.

At 620, the method 600 includes updating the one or more visual indicators during a current contraction. The one or more visual indicators comprises at least the first visual indicator that updates based on a current progression of labor, a second visual indicator that updates based on the extent of cervical dilation, a third visual indicator for displaying a message to the patient and updates based on a fourth visual indicator, and the fourth visual indicator that updates to indicate a remaining time for pushing and a resting time for the patient. In some examples, one or more audio indicators may be generated and output to the patient in response to one of the first visual indicator, the second visual indicator, the third visual indicator, and the fourth visual indicator being updated.

The first visual indicator may update in response to receiving additional labor-related data that results in the second visual indicator being updated and/or other labor-related data including cervical effacement, fetal station, etc. The second visual indicator is updated independent of the first visual indicator, the third visual indicator, and the fourth visual indicator. In one example, the second visual indicator depicting the extent of cervical dilation may be updated in response to receiving user input regarding manually-obtained cervimetry data. Further, the UA detection system may be coupled to a digital cervimetry device. As such, the second visual indicator may be updated based on cervical dilation data received from the digital cervimetry device.

For a duration of the current contraction, the fourth visual indicator may be updated to depict the remaining time and the updated fourth visual indicator may be displayed to the patient via the display device. At the end of the current contraction the third visual indicator may be updated to prompt the patient to stop pushing and the fourth visual indicator may be updated to depict there that there is no remaining time for pushing as illustrated in FIG. 10. In this way, the updated third visual indicator and the updated fourth visual indicator may be displayed to the patient, and a second audio indicator may be generated and output to alert the patient to stop pushing.

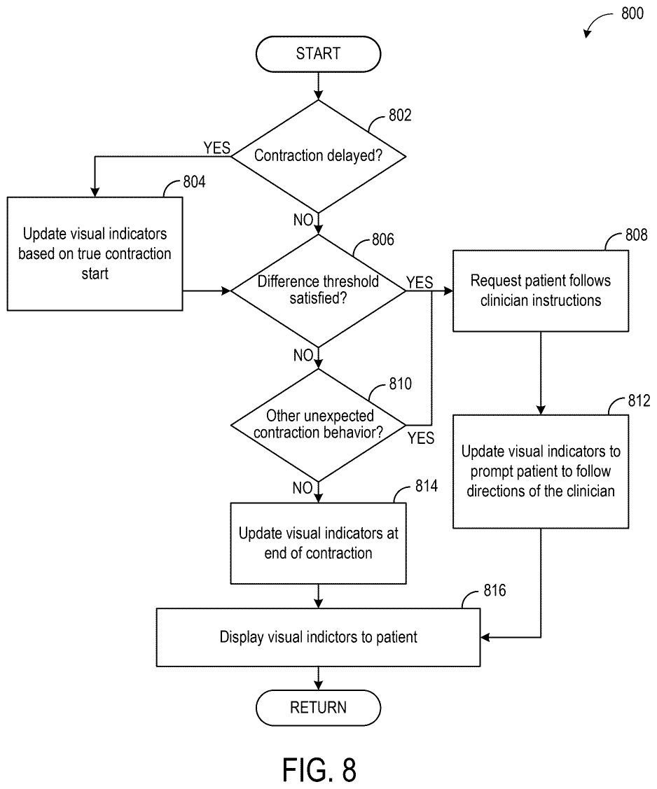

The one or more visual indicators may also update in response to receiving user input. For example, the user input may initiate a manual override of the methods described herein that cause the third visual indicator and the fourth visual indicator to update, which in turn, may prompt the patient to enter the resting time (e.g., FIG. 10) or the follow directions of the clinician (e.g., FIG. 12). The method described in FIG. 8 depicts alternative ways in which the one or more visual indicators may be updated. The method 600 then returns. It may be understood that the method 600 may be performed for each contraction. It may be understood that the method 600 may be used for subsequent contractions as well.

FIG. 7 illustrates a method 700 for generating one or more visual indicators during a start of a next contraction. The method 700 described herein may be executed by a processor of a UA detection system, such as UA detection system 202 of FIG. 2. Operations of the method 700 may be stored in non-transitory memory of the UA detection system (e.g., in a labor coach module of the UA detection system 202 of FIG. 2) and executed by a processor of the UA detection system (e.g., processor 204).

A 702, the method 700 includes determining whether obtained UA data is intermittent or irregular. The UA data may be considered intermittent if a first pre-determined number of data points for a contraction of a particular duration are not received by the UA detection system (e.g., there are gaps in the UA data received). The first pre-determined number of data points may differ based on the duration of the respective contraction and/or the type of UA data. Additionally, the UA data may be considered intermittent if there are not a pre-determined number of contractions for a pre-determined duration of time at a particular point of labor progression. For example, the UA data may be considered intermittent if no UA data is received during a ten minute period.

Contractions with insufficient data points may be unreliable for predicting contraction data using the labor prediction model described herein with respect to FIG. 3B, and 4-6. However, intermittent UA data may still be used to predict contraction data using the labor prediction model depending on whether a second pre-determined number of data points for a contraction of a particular length are obtained, the second predetermined number of data points being less than the first pre-determined number of data points. Alternatively, the UA data may be considered irregular if the UA data deviates from expected UA data values.

In response to determining that the obtained UA data being intermittent or irregular, the method 700 includes determining whether the UA data quantity or regularity threshold is satisfied at 704. The UA data quantity threshold may be satisfied when the number of obtained UA data points is greater than the second pre-determined number of data points for a contraction of a particular duration. The second pre-determined number of data points may differ based on the duration of the respective contraction and/or the type of UA data. The UA data may be considered irregular if a pre-determined regularity threshold is not satisfied. Accordingly, when the number of obtained UA data points is greater than the second pre-determined number of data points or the UA data regularity threshold is satisfied, there is sufficient data within an accepted regularity to perform UA data imputation methods and UA prediction methods. Otherwise, there is not sufficient data (e.g., the UA data gaps are unacceptably large and/or the UA data is unacceptably irregular) to perform data imputation on the UA data and/or predict UA data.

In response to determining that the UA data quantity threshold or UA data regularity threshold not being satisfied, the method 700 includes requesting the patient to follow clinician instructions at 710. The UA data quantity threshold not being satisfied and the UA data regularity threshold not being satisfied may indicate that predictions for a next contraction may be unreliable based on not having a sufficient quantity of UA data or the UA data not being sufficiently regular to enter into the labor prediction model. As such, the clinician assesses the state of the patient based on the UA data for the next contraction and provides instructions to the patient during the next contraction.

At 712, the method 700 includes generating visual indicators and/or audio indicators and outputting the visual indicators and/or audio indicators to a display device. At least a first visual indicator for a progression of labor, a second visual indicator for an extent of cervical dilation, and a third visual indicator that updates in real-time and relays a message to the patient may be generated. The third visual indicator, in particular, may prompt the patient to follow the instructions of the clinician during the next contraction. An example of the third visual indicator prompting the patient to follows instructions of the clinician is depicted in FIG. 12. The at least first visual indicator, the second visual indicator, and the third visual indicator may be displayed on a display device visible to the patient. The method 700 then ends.

In response to the UA data quantity threshold and the UA data regularity threshold being satisfied, the method 700 includes performing imputation on the UA data or predicting UA data at 706. Data imputation may be performed on the UA data using linear interpolation and/or Hermite spline interpolation. Alternatively, the UA data may be predicted by using averaging algorithms. In this way, more accurate values may be output from the labor prediction ML/DL model compared to output obtained with missing UA data due to compensating for a lack of available data (e.g., gaps in the available UA data).

In response to one of the UA data not being intermittent and/or irregular, and the UA data being imputated or predicted, the method 700 includes entering the UA data, the labor-related data, the predicted UA data, and the predicted labor-related data into a labor prediction ML/DL model trained on UA data at 708. Accordingly, contraction data may be output from the labor prediction ML/DL model trained on UA data and other labor-related data. The output from the labor prediction ML/DL model may include the demanded time for pushing during the next contraction. This particular output may be used to generate the fourth visual indicator as described herein.

At 712, the method 700 includes generating visual indicators and/or audio indicators and outputting the visual indicators and/or audio indicators to the display device. The labor contraction data from the labor prediction model may be used to generate the first visual indicator, the second visual indicator, the third visual indicator, the fourth visual indicator, and any audio indicators. The method 700 then ends.

FIG. 8 illustrates a method 800 for updating one or more visual indicators during a current contraction. The method 800 described herein may be executed by a processor of a UA detection system, such as UA detection system 202 of FIG. 2. Operations of the method 800 may be stored in non-transitory memory of the UA detection system (e.g., in a labor coach module of the UA detection system 202 of FIG. 2) and executed by a processor of the UA detection system (e.g., processor 204).

At 802, the method 800 includes determining whether the next contraction is delayed. As described herein with respect to FIGS. 3A, 6, and 7, predicted UA data and predicted labor-related data may be generated by entering UA data and labor-related data for one or more previous contractions into a contraction prediction algorithm or with one or more contraction prediction models (e.g., contraction prediction models 340). In some examples, the predicted start of the next contraction may be determined by a contraction frequency of the one or more previous contractions and the predicted contraction frequency. The next contraction may be considered delayed if the current contraction is delayed compared to the predicted start time of the next contraction, the next contraction being the most recent contraction.

In response to the next contraction being delayed, the method 800 includes updating one or more visual indicators based on the true contraction start at 804. The generation of the fourth visual indicator, an update to the fourth visual indicator, and the update to the third visual indicator may be delayed in response to the start time of the next contraction being delayed compared to the predicted start time of the next contraction. Prior to the start of the next contraction, the display device may display at least the first visual indicator, the second visual indicator, and the third visual indicator to the patient, the first visual indicator depicting a progression of labor, the second visual indicator depicting an extent of cervical dilation, and the third visual indicator depicting instructions for the patient.

In some examples, the third visual indicator may depict a message prompting the patient to follow directions of the clinician and a fourth visual indicator may not be displayed to the patient as illustrated in FIG. 12. In other examples, the third visual indicator may depict a message prompting the patient to not push for a period of time and the fourth visual indicator may not depict a pushing timer as illustrated in FIG. 10. Once the UA data for the current contraction is being collected, and thus the current contraction starts, the fourth visual indicator may be generated or updated and the third visual indicator may be updated to instruct the patient to start pushing as shown in FIG. 13.

In response to the next contraction not being delayed, the method 800 includes determining whether a difference threshold is satisfied at 806. Since the next contraction is not delayed, the third visual indicator may update to prompt the user to start pushing and the fourth visual indicator may be updated to alert the patient to start pushing. As the contraction progresses, the fourth visual indicator may continue to update to visually and textually depict the remaining time for pushing as illustrated in FIG. 13.

As described herein with respect to FIGS. 3A, 5, and 6, predicted UA data and predicted labor-related data for a next contraction may be generated by entering UA data and labor-related data for one or more previous contractions into the contraction prediction algorithm or into one or more contraction prediction models (e.g., contraction prediction models 340). In some examples, as the contraction progresses, the labor-related data may differ significantly from the predicted labor-related data. As such, the labor-related data may be considered significantly different from the predicted labor-related data in response to a difference threshold not being satisfied. In one example, the duration of the next contraction may be determined based on the duration of the one or more previous contractions. The next contraction may be considered prolonged if the next contraction is prolonged compared to the predicted duration of the next contraction, the next contraction being the most recent contraction. As such, the difference threshold for the contraction duration may not be satisfied in cases wherein a true contraction duration is significantly longer than the predicted contraction duration.

In response to the difference threshold not being satisfied, the method 800 includes requesting the patient to follow clinician instructions at 808. The difference threshold not being satisfied may indicate that the progression of labor is abnormal and predictions for a subsequent contraction may be unreliable. As such, the clinician assesses the state of the patient based on the UA data for the next contraction and provides instructions to the patient during the next contraction.

At 812, the method 800 includes updating the one or more visual indicators to prompt the patient to follow the directions of the clinician. Prior to the difference threshold not being satisfied, the third visual indicator may prompt the patient to push and the fourth visual indicator may visually and textually depict the amount of time remaining as shown in FIG. 11. In response to difference threshold not being satisfied, the third visual indicator may be updated to prompt the patient to follow the directions of the clinician and the fourth visual indicator may be updated such that the fourth visual indicator ceases to be displayed to the patient as shown in FIG. 12.

At 816, the method 800 includes displaying the one or more visual indicators to the patient. The one or more visual indicators may be displayed on a display device visible to the patient. In this way, the updated visual indicators may alert the patient to follow the direction of the clinician. The method 800 then returns.

In response to the difference threshold being satisfied, the method 800 includes determining whether there is any unexpected contraction behavior at 810. As mentioned herein, the next contraction may be considered abnormal when the UA data collected during the contraction and the predicted UA data are not within accepted UA data thresholds and the other labor-related data and the predicted labor-related data are not within accepted labor-related data thresholds. The next contraction may be considered abnormal when the acquired labor-related data differs from the predicted labor-related data. In some examples, as the current contraction progresses and labor-related data is obtained (e.g., the UA data and the other labor-related data), the UA data and the other labor-related data may no longer satisfy their respective thresholds.

In response to the next contraction having unexpected contraction behavior, the method 800 includes requesting the patient to follow clinician instructions at 808. The unexpected contraction behavior may indicate that the progression of labor is abnormal and predictions for a subsequent contraction may be unreliable. As such, the clinician assesses the state of the patient based on the UA data for the next contraction and provides instructions to the patient during the next contraction and potentially any subsequent contractions.

At 812, the method 800 includes updating the one or more visual indicators to prompt the patient to follow the directions of the clinicians. Prior to the next contraction having unexpected contraction behavior, the third visual indicator may prompt the patient to push and the fourth visual indicator may visually and textually depict the amount of time remaining as shown in FIG. 11. In response to the next contraction having unexpected contraction behavior, the third visual indicator may be updated to prompt the patient to follow the directions of the clinician and the fourth visual indicator may be updated such that the fourth visual indicator ceases to be displayed to the patient as shown in FIG. 12.

At 816, the method 800 includes displaying the one or more visual indicators to the patient. The one or more visual indicators may be displayed on a display device visible to the patient. In this way, the updated visual indicators may alert the patient to follow the direction of the clinician. The method 800 then returns.

In response to the next contraction not having unexpected contraction behavior, the method 800 includes updating the one or more visual indicators at the end of the contraction at 814. In one example, the fourth visual indicator may update to depict a most recent estimate for the remaining amount of time during the contraction. In another example, the third visual indicator may update to prompt the patient to cease pushing and the fourth visual indicator may update such that the fourth visual indicator ceases to visually and textually depict the remaining time for pushing as shown in FIG. 12. Further, a second audio indicator may be output to the patient via a speaker coupled to or integrated within the display device. In this way, the patient may be audibly alerted to the contraction ending.

At 816, the method 800 includes displaying the one or more visual indicators to the patient. The one or more visual indicators may be displayed on a display device visible to the patient. In this way, the updated visual indicators may alert the patient to continue pushing as shown in FIG. 11 or to cease pushing and to enter a resting time as shown in FIG. 12. The method 800 then returns.

It may be understood that the method 800 may be used for subsequent contraction as well. Although FIG. 8 describes an example for delayed start of contraction, changes in contraction duration may also be calculated during the contraction itself based on the rise time and the maximum strength, for example. The display may be updated to reflect the changes in contraction duration as well.

Referring now to FIG. 9, a flowchart is shown of a method 900 for training a labor prediction model. The labor prediction model may be a non-limiting example of the labor prediction model 418 of the process 400 of FIG. 4, according to an embodiment. In some embodiments, the labor prediction model may be one or more machine learning (ML) or deep learning (DL) models. Method 900 may be executed by a processor of a UA detection system, such as the UA detection system 202 of FIG. 2. Method 900 may be carried out according to instructions stored in non-transitory memory of the UA detection system (e.g., in a training module such as the training module 220 of FIG. 2) and executed by a processor of the UA detection system (e.g., the processor 204 of UA detection system 202 of FIG. 2).

The labor prediction ML/DL model may be trained on training data comprising one or more sets of labor-related data for different patients. Each set of the one or more sets of labor-related data may include contraction data for a plurality of contractions during labor, as described below. In some embodiments, the one or more sets of labor-related data may be stored in a labor-related database of an UA detection system, such as the UA time-series data 210, the maternal parameters data 212, the fetal parameters data 214, and the labor parameters data of UA detection system 202 of FIG. 2, which may include the UA data quantity parameter(s).

At 902, the method 900 includes receiving a ground truth labor-related training dataset, the ground truth labor-related dataset being ground truth contraction data. The ground truth labor-related training dataset may include UA training data (e.g., toco training data, EHG training data, IUPC training data, etc.) for a plurality of patients and other labor-related training data for the plurality of patients. The other labor-related training data may include number of previous pregnancies for each patient, number of C-sections for each patient, MHR and BP during each contraction for each patient, FHR during each contraction for each patient, rise times for each contraction for each patient, and the like. The ground truth labor-related training dataset include labels indicating whether the UA data is intermittent and unusable, whether the UA data is intermittent and usable, whether the labor-related data is conducive for pushing, whether the labor-related data is conducive for clinician intervention, and estimated contraction data including progression of labor, extent of cervical dilation, and a pushing time for the next contraction. The ground truth labor-related training dataset may be stored the UA detection system (e.g., FIG. 2).

At 904, the method 900 includes sorting the ground truth labor-related dataset into datasets for each patient. As described above, the ground truth labor-related training dataset comprises data collected for a plurality of patients during labor. Each subset of ground truth labor-related training data includes training data pertaining to one patient during labor. In one embodiment, each subset of ground truth labor-related training data may correspond to a different patient.

At 906, the method 900 includes selecting one subset of ground truth labor-related training data corresponding to one patient. Each ground truth labor-related training dataset for a patient may include relevant contraction data for each contraction during labor. For example, the ground truth labor-related training dataset may indicate whether the UA data is intermittent and unusable for each contraction, whether the UA data is intermittent and usable for each contraction, whether the labor-related data is conducive for clinician intervention (e.g., outside the accepted thresholds for each contraction), and estimated contraction data including progression of labor, extent of cervical dilation, and a pushing time for each contraction.

At 908, the method 900 includes selecting ground truth labor-related training data corresponding to at least one pair of contractions. In one example, the at least one pair contractions may be one pair of contractions wherein one contraction is considered the previous contraction and the other contraction is considered the next contraction. In other examples, the at least one pair of contractions may be one or more contractions wherein at least one contraction is considered the next contraction and two or more contractions are considered the previous contractions. Instructions configured, stored, and executed in memory by a processor may cause the processor to randomly select the contractions. Alternatively, instructions configured, stored, and executed in memory by a processor may cause process to select the contractions based on a time stamp.

At 910, the method 900 includes inputting the selected set of ground truth labor-related training data into the labor prediction ML/DL model. The labor prediction model ML/DL model is trained on UA data, including toco data, EHG data, and IUPC data, and other labor-related data. In this way, depending on the type of UA data available, contraction data may be estimated using different types of UA data with the labor prediction ML/DL model.

At 912, the method 900 includes receiving contraction data from the labor prediction ML/DL model. The labor prediction ML/DL model may output a set of contraction data for a next contraction included in the ground truth labor-related training dataset.

At 914, the method 900 includes comparing the ground truth contraction data and output contraction data and adjusting model parameters of the labor prediction ML/DL model. More specifically, the ground truth contraction data of the ground truth labor-related training dataset may be compared with the set of contraction data output from the labor prediction ML/DL model. In an example, a loss function of the labor prediction ML/DL model may be determined based on the ground truth contraction data of the ground truth labor-related training dataset and the set of output contraction data for the next contraction. As such, the loss function of the labor prediction ML/DL model may be used to update the parameters of the labor prediction ML/DL model.

At 916, the method 900 includes determining whether additional contractions remain in the ground truth labor-related training data. The plurality of contractions included in the ground truth labor-related training data may include a pre-determined number of next contractions. If less than all of the pre-determined number of next contractions have been selected and used to train the labor prediction ML/DL model (e.g., at least some contractions and corresponding contraction data remain), or if the labor prediction ML/DL model is otherwise determined to not be fully trained, method 900 returns to 908 to select ground truth labor-related training data corresponding to a next at least one pair of contractions and use the next contraction and corresponding contraction training data to train the labor prediction ML/DL model. However, if at 916 it is determined that each contraction and corresponding contraction training data has been selected and used to train the labor prediction ML/DL model (and no more contractions and corresponding contraction training data remain), or if the labor prediction ML/DL model is otherwise determined to be fully trained, method 900 proceeds to 918, which includes determining whether additional subsets of ground truth labor-related training data remain.

The plurality of subsets of labor-related data may include a pre-determined number of subsets of labor-related data for a plurality of patients. If less than all of the pre-determined number of subsets have been selected and used to train the labor prediction ML/DL model (e.g., at least some subsets remain), or if the labor prediction ML/DL model is otherwise determined to not be fully trained, method 900 returns to 906 to select a next subset and use the next subset to train the labor prediction ML/DL model. However, if at 918 it is determined that each subset has been selected and used to train the labor prediction ML/DL model (and no more subsets remain), or if the labor prediction ML/DL model is otherwise determined to be fully trained, the method 900 then returns.

FIG. 13 depicts a timing diagram 1300 illustrating updates to one or more visual indicators displayed on a display device, such as display device 234, during a contraction according to the method described herein. At time t0, at the start of the current contraction, the display device outputs the display output 1302. As described herein, the display output 1302 includes the first label 1002 for the first visual indicator 1004 that depicts the progression of labor, a second label 1006 for the second visual indicator 1008 that depicts the extent of cervical dilation of the patient, and the third label 1010 for the third visual indicator 1012 and the fourth visual indicator 1014. The third visual indicator 1012 includes written text that prompts the patient to start pushing and the fourth visual indicator 1014 visually and textually prompts the patient to start pushing as well.

At first time t1 during the current contraction, the display device outputs the display output 1304. The first label 1002, the second label 1006, the third label 1010, the first visual indicator 1004, and the second visual indicator 1008 remain the same in the display output 1304. However, the third visual indicator 1012 and the fourth visual indicator are updated in the display output 1304 compared to the display output 1302. More specifically, the third visual indicator 1012 includes written text prompting the patient to continue pushing and the fourth visual indicator 1014 visually and textually depicts the amount of remaining time for pushing.

At a second time t2 during the current contraction, the display device outputs the display output 1306. The first label 1002, the second label 1006, the third label 1010, the first visual indicator 1004, and the second visual indicator 1008 remain the same in the display output 1304. However, the third visual indicator 1012 and the fourth visual indicator are updated in the display output 1306 compared to the display output 1304. In particular, the third visual indicator 1012 includes written text prompting the patient to continue pushing and the fourth visual indicator 1014 visually and textually depicts a different amount of remaining time for pushing.

The technical effect of generating visual cues in the form of visual indicators and audio indicators based on output from the labor prediction model that utilize labor-related data for one or more previous contraction and predicted labor-related data for a next contraction and subsequent contractions is that appropriately timed pushing and a desired progression of labor may occur due to more accurate pushing instructions being generated based on the output from the labor prediction model. The pushing instructions may be more accurate due to generating instructions based on UA data acquired from different modalities and the medical history of the patient, including a number of previous pregnancies, C-sections, and other previous labor-related or pregnancy-related complications Accordingly, undesired labor progression or labor characteristics may be reduced by providing the most accurate information with regards to pushing timing to the patient, which may reduce labor progression time and increase labor efficacy.

The disclosure also provides support for a method, comprising: receiving labor-related data for one or more previous contractions of a patient, the labor-related data at least comprising data obtained with one of a tocodynamometer, an IUPC, and an EHG recording system, predicting labor-related data for a next contraction based on the labor-related data for one or more previous contractions, generating one or more visual indicators and one or more audio indicators based on output from a labor prediction model, the output from the labor prediction model being generated by entering the received labor-related data for the one or more previous contractions, the predicted labor-related data, and user input into the labor prediction model, and displaying the one or more visual indicators to a display device that is visible to the patient and outputting the one or more audio indicators to the patient.

In a first example of the method, the labor-related data further comprises maternal parameters data, fetal parameters data, and labor parameters data. In a second example of the method, optionally including the first example, the maternal parameters data includes a number of previous pregnancies, a number of previous C-sections, maternal heart rate (MHR), blood pressure, and the like. In a third example of the method, optionally including one or both of the first and second examples, the fetal parameters data includes fetal heart rate (FHR) and fetal movement. In a fourth example of the method, optionally including one or more or each of the first through third examples, the labor parameters data includes an acceptable number of contractions per time, an acceptable contraction duration, an acceptable contraction strength, unacceptable rise times for a contraction, and indication that labor is progressing. In a fifth example of the method, optionally including one or more or each of the first through fourth examples, predicting labor-related data for the next contraction based on the labor-related data for one or more previous contractions comprises generating predicted labor-related data for the next contraction by entering the labor-related data into a contraction prediction model or contraction prediction algorithm.

In a sixth example of the method, optionally including one or more or each of the first through fifth examples, the contraction prediction model is one or more ML/DL models trained on UA time-series data obtained with one of the IUPC, the tocodynamometer, or the EHG recording system. In a seventh example of the method, optionally including one or more or each of the first through sixth examples, the output from the labor prediction model includes a progression of labor, an extent of cervical dilation, and determining an anticipated pushing time, an anticipated start time for pushing, and an anticipated end time. In an eighth example of the method, optionally including one or more or each of the first through seventh examples, generating one or more visual indicators based on output from the labor prediction model, comprises: generating a first visual indicator that depicts the progression of labor, generating a second visual indicator that depicts the extent of cervical dilation of the patient, and generating a third visual indicator that updates in real-time and displays instructions for the patient.

In a ninth example of the method, optionally including one or more or each of the first through eighth examples, in response to one of the received labor-related data and the predicted labor-related data not being within labor-related data thresholds, the third visual indicator displays instructions prompting the patient to follow directions of a clinician. In a tenth example of the method, optionally including one or more or each of the first through ninth examples, in response to the received labor-related data and the predicted labor-related data being within labor-related data thresholds, generating one or more visual indicators based on the received labor-related data for the one or more previous contractions, the predicted labor-related data, and user input further comprises generating a fourth visual indicator that updates in real-time and depicts a start time for pushing, a remaining time for pushing, and a resting time for the patient and updating the third visual indicator based on the fourth visual indicator.

In an eleventh example of the method, optionally including one or more or each of the first through tenth examples, a generation of the fourth visual indicator, an update to the fourth visual indicator, and an update to the third visual indicator is delayed in response to a start time of the next contraction being delayed compared to a predicted start time of the next contraction. In a twelfth example of the method, optionally including one or more or each of the first through eleventh examples, the third visual indicator is updated to display instructions prompting the patient to follow directions of a clinician and the fourth visual indicator ceases to be displayed to the patient in response to a difference threshold for the labor-related data and the predicted labor-related data not being satisfied.

The disclosure also provides support for a UA detection system, comprising: a display device that outputs one or more visual indicators to a patient during labor, wherein the UA detection system is coupled to one or more of an IUPC, a tocodynamometer, and an EHG recording system, and a processor communicably coupled to the display device, and a non-transitory memory including instructions that when executed cause the processor to: receive labor-related data for one or more previous contractions, the labor-related data at least comprising data obtained with one or more of the tocodynamometer, the IUPC, and the EHG recording system, predict labor-related data for a next contraction based on the labor-related data for one or more previous contractions, generate one or more visual indicators based on output from the labor prediction model, the output from the labor prediction model being generated by entering the received labor-related data for the one or more previous contractions, the predicted labor-related data, and user input into the labor prediction model, display one or more visual indicators to the display device that is visible to the patient. In a first example of the system, the user input includes manual cervimetry data and the user input initiates a manual override that causes a third visual indicator for displaying a message to the patient and a fourth visual indicator that updates to indicate a start time for pushing, a remaining time for pushing, a resting time for the patient to update, prompting the patient to enter the resting time or to follow directions of a clinician. In a second example of the system, optionally including the first example, the UA detection system is coupled to a digital cervimetry device and wherein a second visual indicator updates based on cervical dilation data received from the digital cervimetry device or manual cervimetry data. In a third example of the system, optionally including one or both of the first and second examples, UA data imputation using linear interpolation or Hermite spline interpolation and UA data prediction may be performed in response to receiving intermittent or irregular UA data.

The disclosure also provides support for a method, comprising: extracting UA data for one or more previous contractions of a patient, determining UA data and other labor-related data for one or more previous contractions, in response to the UA data not being within UA data thresholds or the other labor-related data not being within labor-related data thresholds, requesting the patient to follow clinician instructions and displaying one or more visual indicators on a display device to the patient at a start of a next contraction, predicting UA data and other labor-related data for the next contraction with a contraction prediction algorithm or contraction prediction model, in response to the predicted UA data not being within UA data thresholds or the predicted labor-related data not being within labor-related data thresholds, requesting the patient to follow clinician instructions and displaying one or more visual indicators on the display device to the patient at the start of the next contraction, in response to the predicted UA data being within UA data thresholds or the predicted labor-related data being within labor-related data thresholds, displaying one or more visual indicators on the display device to the patient at the start of the next contraction, and updating one or more visual indicators during current contraction.

In a first example of the method, the one or more visual indicators are generated based on output from a labor prediction model and training of the labor prediction model comprises: receiving a ground truth labor-related training dataset for a plurality of patients, each ground truth labor-related training dataset including ground truth contraction data, sorting the ground truth labor-related training dataset into training datasets for each patient, for each patient, selecting one subset of ground truth labor related training data corresponding to one patient, for each contraction, selecting ground truth labor-related training data corresponding to at least one pair of contractions, inputting the selected set of ground truth labor-related training data into a labor prediction model, receiving contraction data for a next contraction from the labor prediction model, and comparing the ground truth contraction data and the output contraction data and adjust model parameters based on the comparison. In a second example of the method, optionally including the first example, the labor prediction model is trained on IUPC data, toco data, EHG data, and other labor-related data.

When introducing elements of various embodiments of the present disclosure, the articles “a,” “an,” and “the” are intended to mean that there are one or more of the elements. The terms “first,” “second,” and the like, do not denote any order, quantity, or importance, but rather are used to distinguish one element from another. The terms “comprising,” “including,” and “having” are intended to be inclusive and mean that there may be additional elements other than the listed elements. As the terms “connected to,” “coupled to,” etc. are used herein, one object (e.g., a material, element, structure, member, etc.) can be connected to or coupled to another object regardless of whether the one object is directly connected or coupled to the other object or whether there are one or more intervening objects between the one object and the other object. In addition, it should be understood that references to “one embodiment” or “an embodiment” of the present disclosure are not intended to be interpreted as excluding the existence of additional embodiments that also incorporate the recited features.

In addition to any previously indicated modification, numerous other variations and alternative arrangements may be devised by those skilled in the art without departing from the spirit and scope of this description, and appended claims are intended to cover such modifications and arrangements. Thus, while the information has been described above with particularity and detail in connection with what is presently deemed to be the most practical and preferred aspects, it will be apparent to those of ordinary skill in the art that numerous modifications, including, but not limited to, form, function, manner of operation and use may be made without departing from the principles and concepts set forth herein. Also, as used herein, the examples and embodiments, in all respects, are meant to be illustrative and should not be construed to be limiting in any manner.

1. A method, comprising:
receiving labor-related data for one or more previous contractions of a patient, the labor-related data at least comprising data obtained with one of a tocodynamometer, an IUPC, and an EHG recording system;
predicting labor-related data for a next contraction based on the labor-related data for one or more previous contractions;
generating one or more visual indicators and one or more audio indicators based on output from a labor prediction model, the output from the labor prediction model being generated by entering the received labor-related data for the one or more previous contractions, the predicted labor-related data, and user input into the labor prediction model; and
displaying the one or more visual indicators to a display device that is visible to the patient and outputting the one or more audio indicators to the patient.

2. The method of claim 1, wherein the labor-related data further comprises maternal parameters data, fetal parameters data, and labor parameters data.

3. The method of claim 2, wherein the maternal parameters data includes a number of previous pregnancies, a number of previous C-sections, maternal heart rate (MHR), blood pressure, and the like.

4. The method of claim 2, wherein the fetal parameters data includes fetal heart rate (FHR) and fetal movement.

5. The method of claim 2, wherein the labor parameters data includes an acceptable number of contractions per time, an acceptable contraction duration, an acceptable contraction strength, unacceptable rise times for a contraction, and indication that labor is progressing.

6. The method of claim 1, wherein predicting labor-related data for the next contraction based on the labor-related data for one or more previous contractions comprises generating predicted labor-related data for the next contraction by entering the labor-related data into a contraction prediction model or contraction prediction algorithm.

7. The method of claim 6, wherein the contraction prediction model is one or more ML/DL models trained on UA time-series data obtained with one of the IUPC, the tocodynamometer, or the EHG recording system.

8. The method of claim 1, wherein the output from the labor prediction model includes a progression of labor, an extent of cervical dilation, and determining an anticipated pushing time, an anticipated start time for pushing, and an anticipated end time.

9. The method of claim 8, wherein generating one or more visual indicators based on output from the labor prediction model, comprises:
generating a first visual indicator that depicts the progression of labor;
generating a second visual indicator that depicts the extent of cervical dilation of the patient; and
generating a third visual indicator that updates in real-time and displays instructions for the patient.

10. The method of claim 9, wherein in response to one of the received labor-related data and the predicted labor-related data not being within labor-related data thresholds, the third visual indicator displays instructions prompting the patient to follow directions of a clinician.

11. The method of claim 9, wherein in response to the received labor-related data and the predicted labor-related data being within labor-related data thresholds, generating one or more visual indicators based on the received labor-related data for the one or more previous contractions, the predicted labor-related data, and user input further comprises generating a fourth visual indicator that updates in real-time and depicts a start time for pushing, a remaining time for pushing, and a resting time for the patient and updating the third visual indicator based on the fourth visual indicator.

12. The method of claim 11, wherein a generation of the fourth visual indicator, an update to the fourth visual indicator, and an update to the third visual indicator is delayed in response to a start time of the next contraction being delayed compared to a predicted start time of the next contraction.

13. The method of claim 11, wherein the third visual indicator is updated to display instructions prompting the patient to follow directions of a clinician and the fourth visual indicator ceases to be displayed to the patient in response to a difference threshold for the labor-related data and the predicted labor-related data not being satisfied.

14. A UA detection system, comprising:
a display device that outputs one or more visual indicators to a patient during labor;
wherein the UA detection system is coupled to one or more of an IUPC, a tocodynamometer, and an EHG recording system; and
a processor communicably coupled to the display device, and a non-transitory memory including instructions that when executed cause the processor to:
receive labor-related data for one or more previous contractions, the labor-related data at least comprising data obtained with one or more of the tocodynamometer, the IUPC, and the EHG recording system;
predict labor-related data for a next contraction based on the labor-related data for one or more previous contractions;
generate one or more visual indicators based on output from the labor prediction model, the output from the labor prediction model being generated by entering the received labor-related data for the one or more previous contractions, the predicted labor-related data, and user input into the labor prediction model;
display one or more visual indicators to the display device that is visible to the patient.

15. The UA detection system of claim 14, wherein the user input includes manual cervimetry data and the user input initiates a manual override that causes a third visual indicator for displaying a message to the patient and a fourth visual indicator that updates to indicate a start time for pushing, a remaining time for pushing, a resting time for the patient to update, prompting the patient to enter the resting time or to follow directions of a clinician.

16. The UA detection system of claim 14, wherein the UA detection system is coupled to a digital cervimetry device and wherein a second visual indicator updates based on cervical dilation data received from the digital cervimetry device or manual cervimetry data.

17. The UA detection system of claim 14, wherein UA data imputation using linear interpolation or Hermite spline interpolation and UA data prediction may be performed in response to receiving intermittent or irregular UA data.

18. A method, comprising:
extracting UA data for one or more previous contractions of a patient;
determining UA data and other labor-related data for one or more previous contractions;
in response to the UA data not being within UA data thresholds or the other labor-related data not being within labor-related data thresholds, requesting the patient to follow clinician instructions and displaying one or more visual indicators on a display device to the patient at a start of a next contraction;
predicting UA data and other labor-related data for the next contraction with a contraction prediction algorithm or contraction prediction model;
in response to the predicted UA data not being within UA data thresholds or the predicted labor-related data not being within labor-related data thresholds, requesting the patient to follow clinician instructions and displaying one or more visual indicators on the display device to the patient at the start of the next contraction;
in response to the predicted UA data being within UA data thresholds or the predicted labor-related data being within labor-related data thresholds, displaying one or more visual indicators on the display device to the patient at the start of the next contraction; and
updating one or more visual indicators during current contraction.

19. The method of claim 18, wherein the one or more visual indicators are generated based on output from a labor prediction model and training of the labor prediction model comprises:
receiving a ground truth labor-related training dataset for a plurality of patients, each ground truth labor-related training dataset including ground truth contraction data;
sorting the ground truth labor -related training dataset into training datasets for each patient;
for each patient, selecting one subset of ground truth labor related training data corresponding to one patient;
for each contraction, selecting ground truth labor-related training data corresponding to at least one pair of contractions;
inputting the selected set of ground truth labor-related training data into a labor prediction model;
receiving contraction data for a next contraction from the labor prediction model; and
comparing the ground truth contraction data and the output contraction data and adjust model parameters based on the comparison.

20. The method of claim 19, wherein the labor prediction model is trained on IUPC data, toco data, EHG data, and other labor-related data.
